(19) United States
(12) Patent Application Publication
(10) Pub. No.: US 2014/0163096 A1
(43) Pub. Date: Jun. 12, 2014

(54) METHODS AND COMPOSITIONS FOR TREATING HIV-ASSOCIATED DIARRHEA

(71) Applicants: Pam Golden, Durham, NC (US); William Forbes, Raleigh, NC (US); Enoch Bortey, Chapel Hill, NC (US)

(72) Inventors: Pam Golden, Durham, NC (US); William Forbes, Raleigh, NC (US); Enoch Bortey, Chapel Hill, NC (US)

(21) Appl. No.: 13/840,861

(22) Filed: Mar. 15, 2013

Related U.S. Application Data
(60) Provisional application No. 61/734,901, filed on Dec. 7, 2012.

(51) Int. Cl. A61K 31/353

(52) U.S. Cl. CPC A61K 31/353
USPC 514/456

(57) ABSTRACT

Provided herein are methods for treating HIV-associated or highly active antiretroviral therapy (HAART)-associated diarrhea in an HIV positive subject by administering a composition comprising crofelemer to the subject wherein the composition has minimal drug-drug interactions with at least one other compound concurrently administered to the subject to treat an HIV infection. Also provided are methods for treating HIV-associated or highly active antiretroviral therapy (HAART)-associated diarrhea in an HIV positive subject by administering a composition comprising crofelemer to the subject, wherein the composition does not significantly inhibit the activity of at least one other compound concurrently administered to the subject to treat an HIV infection.

### RELATED APPLICATIONS

This application claims the benefit of U.S. Provisional Application No. 61/734,901, filed Dec. 7, 2012, which is incorporated herein by reference in its entirety.

### BACKGROUND

Diarrhea remains an important problem for HIV-infected subjects undergoing highly active antiretroviral therapy (HAART) and can have a severely negative impact on quality of life despite the extensive use of anti-diarrheal compounds. The causes of diarrhea in HIV-infected subjects are numerous and include HIV enteropathy, overgrowth of unusual microbial agents, common enteric pathogens malignancy, and adverse effects of HAART therapy itself (Kartalij a 1999).

While definitions and methods of reporting vary, it is estimated that around half of all HIV-AIDS subjects will have diarrhea at some point during their illness. While the introduction of HAART did not appear to significantly impact the incidence of diarrhea in HIV-infected patients, it was observed that the etiologies of diarrhea changed significantly. In particular, an increase of noninfectious causes and a decrease in opportunistic infectious causes of diarrhea was observed in HIV subjects undergoing HAART.

Effective management of diarrhea may assist in improving overall efficacy of anti-viral drug therapies as well as improve quality of life, including controlling weight loss in HIV-positive subjects. Suffering from diarrhea, whether it is associated with HIV infection or with use of antiretroviral (ARV) therapy, may result in reduced compliance with ARV therapy and/or necessitate switching ARV regimens. Diarrhea has also been associated with reduced antiretroviral drug levels, suggesting that adequate treatment of diarrhea may improve the absorption of ARV medication. On a population-wide basis, adherence to drug treatment regimens and maintenance of adequate ARV levels are important for minimizing the development of drug resistant strains of HIV. Therefore, effective management of diarrhea in HIV-positive subjects, whether HIV-related or ARV-related, represents an important and unmet clinical need. Currently prescribed therapies are only partially effective or are plagued by unacceptable side effects such as constipation and the potential for addiction. The development of a composition and methods of using the same for the treatment of HIV-associated diarrhea, where the composition has a low potential for drug-drug interactions, minimal effects on ARV drug metabolism, or minimal abuse potential, would provide an important benefit for HIV-infected subjects.

### SUMMARY OF THE INVENTION

Embodiments are directed to a method of treating HIV-associated or highly active antiretroviral therapy (HAART)-associated diarrhea in an HIV positive subject, wherein the method includes administering a composition comprising crofelemer to the subject, and wherein the composition has minimal drug-drug interactions with at least one other compound concurrently administered to the subject to treat an HIV infection.

Embodiments also relate to a method of treating HIV-associated or highly active antiretroviral therapy (HAART)-associated diarrhea in an HIV positive subject, wherein the method includes administering a composition comprising crofelemer to the subject, and wherein the composition does not significantly inhibit the activity of at least one other compound concurrently administered to the subject to treat an HIV infection.

In some embodiments, the at least one other compound is an anti-retroviral therapy (ART) compound. For example, the at least one other compound can be selected from the group of: ritonavir, tenofovir, emtricitabine, lamivudine, lopinavir and efavirenz.

In some embodiments, the at least one other compound includes efiravenz, emtricitabine and tenofovir. In some embodiments, the at least one other compound includes emtricitabine, ritonavir and tenofovir. In some embodiments, the at least one other compound can further include lopinavir, atazanavir, fosamprenavir, darunavir or a combination thereof.

In some embodiments, the at least one other compound includes emtricitabine, lopinavir, ritonavir and tenofovir.

In some embodiments, the at least one other compound includes lamivudine, lopinavir and ritonavir. In some embodiments, the at least one other compound further includes abacavir, zidovudine or a combination thereof.

In some embodiments, the at least one other compound includes lopinavir and ritonavir. In some embodiments, the at least one other compound further includes emtricitabine, tenofovir, lamivudine, abacavir, zidovudine, didanosine or a combination thereof.

In some embodiments, the at least one other compound includes lamivudine and zidovudine. In some embodiments, the at least one other compound further includes efiravenz, nelfinavir, ritonavir, lopinavir, abacavir or a combination thereof.

Embodiments are also directed to a method of treating HIV-associated or highly active antiretroviral therapy (HAART)-associated diarrhea in an HIV positive subject, wherein the method includes administering a composition comprising crofelemer to the subject, and wherein the composition does not significantly inhibit a CYP enzyme in vivo.

Provided herein is a method of treating HIV-associated or highly active antiretroviral therapy (HAART)-associated diarrhea in an HIV positive subject, wherein the method includes administering a composition comprising crofelemer to the subject, and wherein the composition does not significantly inhibit the activity of a drug transporter in vivo.

Also provided herein is a method of treating HIV-associated or highly active antiretroviral therapy (HAART)-associated diarrhea in an HIV positive subject, wherein the method includes administering a composition comprising crofelemer to the subject, and wherein the composition does not significantly affect the efficacy of at least one other compound concurrently administered to the subject to treat an HIV infection.

Additional embodiments are directed to a method of treating HIV-associated or highly active antiretroviral therapy (HAART)-associated diarrhea in an HIV positive subject, wherein the method includes administering a composition comprising crofelemer to the subject, and wherein the composition has negligible permeability as measured by in vitro permeability assays.

Embodiments also relate to a method of treating HIV-associated or highly active antiretroviral therapy (HAART)-associated diarrhea in an HIV positive subject, wherein the method includes administering a composition comprising crofelemer to the subject, and wherein the composition has limited systemic exposure in vivo.

Embodiments are also directed to a method of treating HIV-associated or highly active antiretroviral therapy (HAART)-associated diarrhea in an HIV positive subject, wherein the method includes administering a composition comprising crofelemer to the subject, and wherein the composition does not provoke a significant adverse event in vivo. In some embodiments, the adverse event is at least one selected from the group of: dyspepsia, flatulence, abdominal pain, hemorrhoids, upper respiratory tract infection, and urinary tract infection.

Also provided herein is a method of treating HIV-associated or highly active antiretroviral therapy (HAART)-associated diarrhea in an HIV positive subject, wherein the method includes administering a composition comprising crofelemer to the subject, and wherein the composition does not cause deterioration of immune status in the subject.

In any of the foregoing embodiments, crofelemer can be administered to the subject at a dosage of about 250 mg per day. In some embodiments, crofelemer is administered to the subject at a dosage of about 125 mg two times per day.

In any of the foregoing embodiments, the composition can be administered to the subject for between about three days and about six months. In some embodiments, the composition is administered to the subject for between about one month and about six months. In some embodiments, the composition is administered to the subject for at least about eight days. In some embodiments, the composition is administered to the subject for between about eight days and about 24 weeks.

In any of the foregoing embodiments, the subject can experience an improvement of symptoms on Day 3 after treatment begins. In some embodiments, administration of the composition results in at least one of the following: an improvement in stool consistency; an improvement in a score for daily stool consistency relative to baseline measurements; alleviation of watery diarrhea; a decrease in the number of bowel movements per day relative to baseline measurements; a decrease in the number of water bowel movements per day relative to baseline measurements; an improvement in the daily abdominal score for pain or discomfort relative to baseline measurements; a decrease in the number of days per week that the subject experiences urgency relative to baseline measurements; and a decrease in the number of days per week that the subject experiences fecal incontinence relative to baseline measurements.

In any of the foregoing embodiments, the composition can be administered to the subject for about six months.

In any of the foregoing embodiments, the composition can be administered to the subject for at least about six months. In some embodiments, the composition is administered to the subject for the duration of the subject's HIV infection.

In any of the foregoing embodiments, the subject can previously have been administered protease inhibitors.

Other embodiments are disclosed infra.

### DESCRIPTION OF THE DRAWINGS

FIG. 1 shows subjects with clinical response in the crossover to placebo-free phase of the safety population.

FIG. 2 shows subjects with stool consistency response in the crossover to placebo-free phase in the safety population.

FIG. 3 depicts a subgroup analysis showing the treatment difference in percentage of responders (crofelemer 125 mg BID vs. placebo) with associated confidence intervals and p-values.

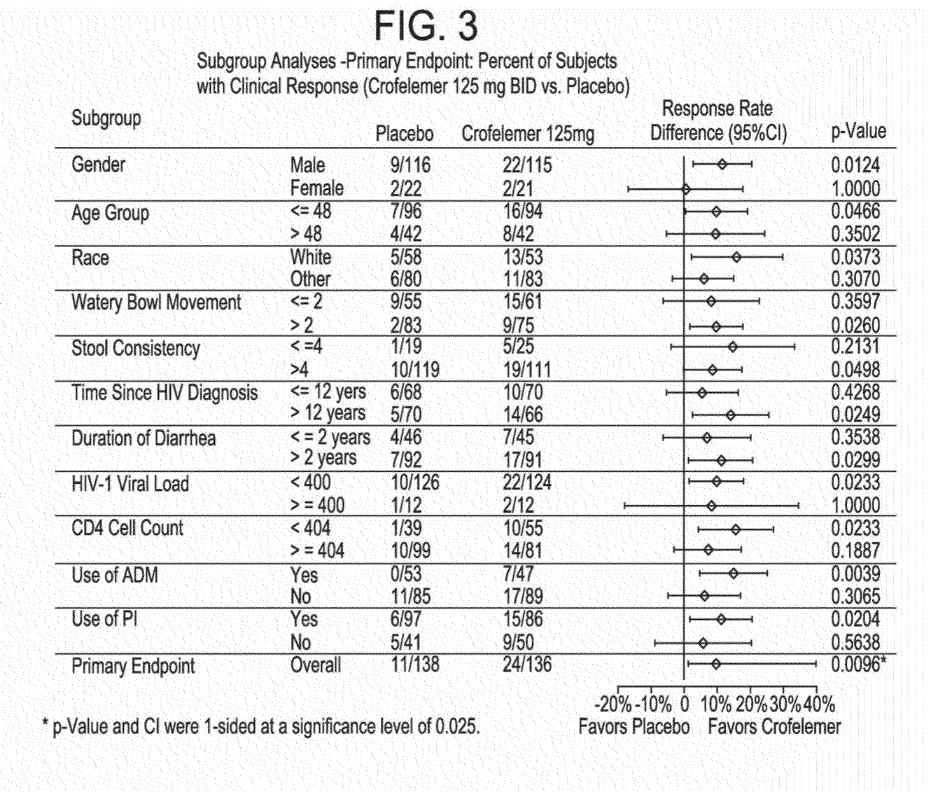

FIG. 4 illustrates a cluster plot of tenofovir concentration ratios overlaid on the same drug concentration ratios without crofelemer treatment (baseline concentration ratios) at Visit 3 of the Phase 3 study described herein.

FIG. 5 illustrates a cluster plot of tenofovir concentration ratios overlaid on the same drug concentration ratios without crofelemer treatment (baseline concentration ratios) at Visit 8 of the Phase 3 study described herein.

FIG. 6 illustrates a cluster plot of ritonavir concentration ratios overlaid on the same drug concentration ratios without crofelemer treatment (baseline concentration ratios) at Visit 3 of the Phase 3 study described herein.

FIG. 7 illustrates a cluster plot of ritonavir concentration ratios overlaid on the same drug concentration ratios without crofelemer treatment (baseline concentration ratios) at Visit 8 of the Phase 3 study described herein.

FIG. 8 illustrates a cluster plot of emtricitabine concentration ratios overlaid on the same drug concentration ratios without crofelemer treatment (baseline concentration ratios) at Visit 3 of the Phase 3 study described herein.

FIG. 9 illustrates a cluster plot of emtricitabine concentration ratios overlaid on the same drug concentration ratios without crofelemer treatment (baseline concentration ratios) at Visit 8 of the Phase 3 study described herein.

FIG. 10 illustrates a cluster plot of lamivudine concentration ratios overlaid on the same drug concentration ratios without crofelemer treatment (baseline concentration ratios) at Visit 3 of the Phase 3 study described herein.

FIG. 11 illustrates a cluster plot of lamivudine concentration ratios overlaid on the same drug concentration ratios without crofelemer treatment (baseline concentration ratios) at Visit 8 of the Phase 3 study described herein.

FIG. 12 illustrates a cluster plot of lopinavir concentration ratios overlaid on the same drug concentration ratios without crofelemer treatment (baseline concentration ratios) at Visit 3 of the Phase 3 study described herein.

FIG. 13 illustrates a cluster plot of lopinavir concentration ratios overlaid on the same drug concentration ratios without crofelemer treatment (baseline concentration ratios) at Visit 8 of the Phase 3 study described herein.

FIG. 14 illustrates a cluster plot of efiravenz concentration ratios overlaid on the same drug concentration ratios without crofelemer treatment (baseline concentration ratios) at Visit 3 of the Phase 3 study described herein.

FIG. 15 illustrates a cluster plot of efiravenz concentration ratios overlaid on the same drug concentration ratios without crofelemer treatment (baseline concentration ratios) at Visit 8 of the Phase 3 study described herein.

### DETAILED DESCRIPTION

The methods disclosed herein involved the administration of effective amounts of a proanthocyanidin polymer, e.g., crofelemer, to subjects having, for example, HIV-associated diarrhea or highly active antiretroviral therapy (HAART)-associated diarrhea.

Proanthocyanidins are a group of condensed tannins. Crude extracts from medicinal plants, for example, Pycanthus angolenis and Baphia nitida, have been shown to have antidiarrheal qualities in animal tests (Onwukaeme and Anuforo, 1993, Discovery and Innovation, 5:317; Onwukaeme and Lot, 1991, Phytotherapy Res., 5:254). Crude extracts which contain tannins, in particular extracts from carob pods and sweet chestnut wood, have been proposed as treatments or prophylactics (U.S. Pat. No. 5,043,160; European Patent No. 481,396).

Proanthocyanidins are comprised of at least two or more monomer units that may be of the same or different monomeric structure. The monomer units (generally termed “leucoanthocyanidin”) are generally monomeric flavonoids which include catechins, epicatechins, gallocatechins, galloepicatechins, flavanols, flavonols, and flavan-3,4-diols, leucocyanidins and anthocyanidins. Therefore, the polymer chains are based on different structural units, which create a wide variation of polymeric proanthocyanidins and a large number of possible isomers (Hemingway et al., 1982, J. Chem. Soc., Perkin Trans., 1:1217). Larger polymers of the flavonoid 3-ol units are predominant in most plants, and are found with average molecular weights above 2,000 daltons, containing 6 or more units (Newman et al., 1987, Mag. Res. Chem., 25:118).

Proanthocyanidin polymers are found in a wide variety of plants, particularly those with a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, found in South America, produce a red viscous latex sap called Sangre de Drago or “Dragon's Blood.” U.S. Pat. No. 5,211,944 first described the isolation of an aqueous soluble proanthocyanidin polymer composition from Croton spp. and the use of the composition as an antiviral agent. (See also Ubillas et al., 1994, Phytomedicine, 1:77). The proanthocyanidin polymer composition was shown to have antiviral activity against a variety of viruses including, respiratory syncytial, influenza, parainfluenza and herpes viruses. U.S. Pat. No. 5,211,944 also discloses the isolation of an aqueous soluble proanthocyanidin polymer composition from Calophyllum inophyllum and the use of this composition as an antiviral agent.

Exemplary proanthocyanidin polymer compositions useful in the methods presented herein are preferably isolated from a Croton spp. or Calophyllum spp. by any method known in the art. For example, the proanthocyanidin polymer composition may be isolated from a Croton spp. or Calophyllum spp. by the method disclosed in U.S. Pat. No. 5,211,944 or in Ubillas et al., 1994, Phytomedicine 1: 77-106, each of which is incorporated herein by reference in its entirety.

Proanthocyanidin polymer compositions useful in the methods presented herein may also be made in vitro using synthetic techniques.

In some embodiments, a proanthocyanidin polymer composition useful in the methods presented herein is crofelemer.

Crofelemer, also known as SP-303, is an oligomeric proanthocyanidin extracted and purified from the red, viscous latex of the plant Croton lechleri of the family Euphorbiace. The plant is widely distributed throughout tropical Central America and South America and is widely recognized by ethnobotanists and local healers for its medicinal properties (McRae et al. 1988. J Ethnopharmacol 22:143-172), including for the treatment of diarrhea. Crofelemer is believed to exert its anti-diarrhea effect through luminal blockade of CFTR (cystic fibrosis transmembrane conductance regulator) chloride (co channels and calcium-activated chloride ion channels (CaCC). Crofelemer has demonstrated in vitro activity against cholera toxin, forskolin, E coli heat-labile (LT) and heat-stable (ST) toxin-mediated Cl⁻ secretion, and to normalize electrolyte and fluid accumulation in cholera toxin-treated mice (Gabriel et al. 1999. Am J Physiol 276 (1 Pt 1):G58-63; Fischer et al. 2004. J Ethnopharmacol 93(2-3):351-357; Adam 2005) via its effects on the CFTR channel. Crofelemer also significantly improved the secretory diarrhea in humans due to enterotoxigenic E. coli (DiCesare et al. 2002. Am J Gastroenterol 97(10):2585-2588), which is also thought to evoke secretory diarrhea through activation of CFTR (Kunzelmann, K. and M. Mall. 2002. Physiol Rev 82:245-289). Blockade of the CFTR channel could be anticipated to have negative consequences in man, even mimicking cystic fibrosis. However, crofelemer has virtually no systemic bioavailability in humans. When studied, the results indicated that there was little or no absorption of crofelemer from the gastrointestinal (GI) tract, and that crofelemer was well tolerated by normal male subjects. Thus, the site of action of crofelemer is topical in the gastrointestinal tract.

Crofelemer (CAS 148465-45-6) is an oligomeric proanthocyanidin of varying chain lengths derived from the Dragon's Blood Croton lecheri of the family Euphorbiaceae. Crofelemer has an average molecular weight of between approximately 1500 daltons and approximately 2900 daltons. The monomers comprising crofelemer include, for example, catechin, epicatechin, gallocatechin, and epigallocatechin, and these monomeric units are linked in random sequence. The chain length of crofelemer ranges from about 3 to about 30 units with an average chain length of about 8 units. The structure of crofelemer is shown below.

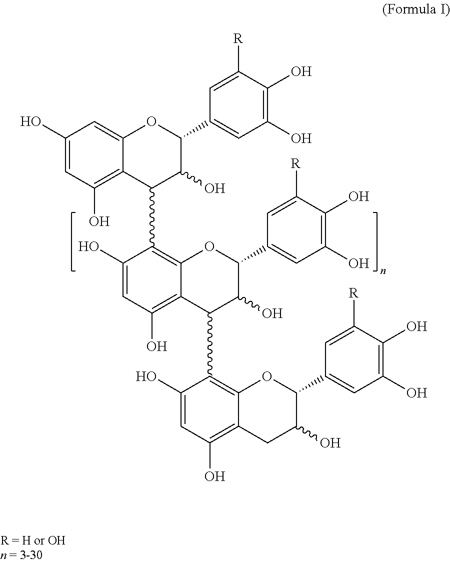

wherein the average n=6.

Another method for isolating crofelemer can be found in U.S. Patent Publication No. 2005/0019389, which is incorporated herein by reference in its entirety.

In some embodiments, a raw latex obtained from a Croton species or a Calophyllum species, or an extract obtained from a Croton species or a Calophyllum species are useful in the methods presented herein. Exemplary extracts are described in Persinos et al., 1979, J. Pharma. Sci. 68:124 and Sethi, 1977, Canadian J. Pharm. Sci. 12:7, each of which is incorporated herein by reference in its entirety.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with crofelemer, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement. Amelioration may be transient, prolonged or permanent or it may be variable at relevant times during or after crofelemer is administered to a subject or is used in an assay or other method described herein or a cited reference, e.g., within timeframes described infra, or about 1 hour after the administration or use of crofelemer to about 7 days, 2 weeks, 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with crofelemer, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively. Modulation may be transient, prolonged or permanent or it may be variable at relevant times during or after crofelemer is administered to a subject or is used in an assay or other method described herein or a cited reference, e.g., within times described infra, or about 1 hour of the administration or use of crofelemer to about 7 days, 2 weeks, 28 days, 3, 6, 9 months or more after a subject(s) has received crofelemer.

The language “a prophylactically effective amount” of a compound refers to an amount of crofelemer which is effective, upon single or multiple dose administration to the subject, in preventing or treating diarrhea, e.g., HIV-associated diarrhea or HAART-associated diarrhea.

As used herein, a “therapeutically effective amount” means an amount of crofelemer effective, when administered to a human or non-human subject, to provide a therapeutic benefit such as an amelioration of symptoms, e.g., an amount effective to decrease the symptoms HIV-associated diarrhea or HAART-associated diarrhea. In some embodiments, a therapeutically effective amount of compound is an amount sufficient to alleviate watery diarrhea, improve stool consistency, decrease the number of bowel movements per day relative to a baseline level, decrease the number of watery bowel movements per day relative to a baseline level, decrease the number of days per week that a subject experiences urgency relative to a baseline level, decrease the number of days per week that a subject experiences fecal incontinence, and/or decrease the severity of diarrheal symptoms (e.g. abdominal pain or discomfort) relative to a baseline level.

As used herein, a “subject” includes an organism which are capable of suffering from HIV-associated diarrhea or HAART-associated diarrhea or who could otherwise benefit from the administration of crofelemer as described herein, such as humans and non-human animals. The term “non-human animals” includes all vertebrates, e.g., mammals such as non-human primates, other mammals, e.g., rodents, and sheep, dog, and cow, at risk for HIV-associated diarrhea or HAART-associated diarrhea. A subject at risk for HIV-associated diarrhea or HAART-associated diarrhea includes a subject at risk of developing or contracting an HIV infection.

The term “administration” or “administering” includes routes of introducing crofelemer to a subject to perform their intended function. Examples of routes of administration that may be used include injection, oral, inhalation, rectal and transdermal. The pharmaceutical preparations may be given by forms suitable for each administration route. For example, these preparations are administered in tablet or capsule form, by injection, inhalation, ointment, or suppository. Administration may also be by injection, infusion or inhalation; topical by lotion or ointment; and rectal by suppositories. Oral administration is preferred. Depending on the route of administration, crofelemer can be coated with or disposed in a selected material to protect it from natural conditions that may detrimentally affect its ability to perform its intended function. Crofelemer can be administered alone, or in conjunction with either another agent or agents as described above or with a pharmaceutically-acceptable carrier, or both. Exemplary enteric coated forms of crofelemer are described in, for example, U.S. Pat. No. 7,556,831.

Administration “in combination with” one or more further therapeutic agents includes simultaneous (concurrent) and consecutive administration in any order.

As will be readily apparent to one skilled in the art, the useful in vivo dosage to be administered and the particular mode of administration may vary depending upon the age, weight and mammalian species treated, the particular compounds employed, and/or the specific use for which these compounds are employed. The determination of effective dosage levels, that is the dosage levels necessary to achieve the desired result, can be accomplished by one skilled in the art using routine pharmacological methods and in consultation with the data presented herein. In regard to crofelemer, it may be advantageous to administer 125 mg crofelemer two times per day to treat watery diarrhea if fewer watery stools are desired over a week. It is also advantageous to treat with 500 mg two times per day if an improvement in stool consistency is desired.

The term “obtaining” as in “obtaining crofelemer” is intended to include purchasing, synthesizing, isolating, extracting or otherwise acquiring crofelemer.

Provided herein are methods of treating, preventing, or alleviating diarrhea or the symptoms caused by HIV infection or HAART therapy for HIV infection comprising administering to a subject in need thereof an effective amount of crofelemer. Examples of diarrhea that can be treated or prevented using the methods presented herein include HIV-associated diarrhea or highly active antiretroviral therapy (HAART)-associated diarrhea.

In some embodiments, treating HIV-associated or HAART-associated diarrhea includes an improvement of symptoms including, for example, a decrease in the number of bowel movements per day (frequency), a decrease in the number of watery bowel movements per day, a decrease in symptom frequency (urgency, fecal incontinence), a decrease in the number of days per week that a subject experiences urgency, a decrease in the number of days per week that a subject experiences fecal incontinence, a decrease in symptom severity (abdominal pain or discomfort), an improvement in the daily abdominal score for pain or discomfort, a decrease or an improvement in daily stool consistency score (watery to formed), a decrease in stool consistency leading to formed stools from watery stools, and/or a decrease in the number of unscheduled visits for a significant worsening of diarrhea.

In some embodiments, treatment results in two or less watery bowel movements per week. In some embodiments, treatment results in two or less watery bowel movements per week during at least two of the four weeks of treatment with crofelemer.

In some embodiments, provided herein are methods of treating HIV-associated diarrhea or highly active antiretroviral therapy (HAART)-associated diarrhea in an HIV positive subject. The methods include, for example, administering a composition comprising crofelemer to the subject. In some embodiments, the composition has minimal drug-drug interaction with at least one other compound or therapy being administered to the subject to treat an HIV infection. In some embodiments, the composition does not significantly inhibit the activity of at least one other compound or therapy being administered to the subject to treat an HIV infection.

In some embodiments, provided herein are methods of treating stool consistency in an HIV positive subject, wherein a subject is considered treated if there is an improvement in the score for daily stool consistency and/or a decrease in stool consistency score a measured throughout the day or days or weeks. This decrease may be measured from a baseline. The baseline may be determined in the days to week prior to treatment with crofelemer. The methods include, for example, administering a composition comprising crofelemer to the subject. In some embodiments, the composition has minimal drug-drug interaction with at least one other compound or therapy being administered to the subject to treat an HIV infection. In some embodiments, the composition does not significantly inhibit the activity of at least one other compound or therapy being administered to the subject to treat an HIV infection.

In some embodiments, provided herein are methods of improving stool consistency in an HIV positive subject wherein a subject is considered treated if there is an improvement in stool consistency and/or a decrease in stool consistency throughout the day or days or weeks. This increase may be measured from a baseline. The baseline may be determined in the days to week prior to treatment with crofelemer. The methods include, for example, administering a composition comprising crofelemer to the subject. In some embodiments, the composition has minimal drug-drug interaction with at least one other compound or therapy being administered to the subject to treat an HIV infection. In some embodiments, the composition does not significantly inhibit the activity of at least one other compound or therapy being administered to the subject to treat an HIV infection.

In some embodiments, provided herein are methods of alleviating watery diarrhea in an HIV positive subject, wherein a subject is considered treated if the subject experiences a decrease in the number of watery bowel movements per day and/or over days, a week or weeks of administration of crofelemer. This decrease may be measured from a baseline. The baseline may be determined in the days to week prior to treatment with crofelemer. The methods include, for example, administering a composition comprising crofelemer to the subject. In some embodiments, the composition has minimal drug-drug interaction with at least one other compound or therapy being administered to the subject to treat an HIV infection. In some embodiments, the composition does not significantly inhibit the activity of at least one other compound or therapy being administered to the subject to treat an HIV infection.

In some embodiments, presented herein are methods a decreasing the number of bowel movements per day, wherein a subject is considered treated if there is a decrease in the number of bowel movements per day as measured from a baseline. The baseline may be determined in the days to week prior to treatment with crofelemer. The methods include, for example, administering a composition comprising crofelemer to the subject. In some embodiments, the composition has minimal drug-drug interaction with at least one other compound or therapy being administered to the subject to treat an HIV infection. In some embodiments, the composition does not significantly inhibit the activity of at least one other compound or therapy being administered to the subject to treat an HIV infection.

In some embodiments, provided herein is a method of treating HIV-associated or highly active antiretroviral therapy (HAART)-associated diarrhea in an HIV positive subject, wherein the method includes administering a composition comprising crofelemer to the subject, wherein the composition has minimal drug-drug interactions with at least one other compound concurrently administered to the subject to treat an HIV infection.

In some embodiments, provided herein is a method of treating HIV-associated or highly active antiretroviral therapy (HAART)-associated diarrhea in an HIV positive subject, wherein the method includes administering a composition comprising crofelemer to the subject, wherein the composition does not significantly inhibit the activity of at least one other compound concurrently administered to the subject to treat an HIV infection.

In some embodiments, provided herein is a method of treating HIV-associated or highly active antiretroviral therapy (HAART)-associated diarrhea in an HIV positive subject, wherein the method includes administering a composition comprising crofelemer to the subject, wherein the composition does not significantly affect the efficacy of at least one other compound concurrently administered to the subject to treat an HIV infection.

In some embodiments, the subject is concurrently being administered an anti-retroviral therapy (ART) compound. In some embodiments, the subject is concurrently being administered at least one selected from the group of: ritanovir, tenofovir, emtricitabine, lamicudine, lopinavir, efavirenz, abacavir, atazanavir, darunavir, didanosine, etravirine, fosamprenavir, indinavir, lamivudine, maraviroc, nelfinavir, nevirapine, raltegravir, saquinavir, stavudine, tipranavir and zidovudine.

In some embodiments, the subject is being administered a combination comprising efiravenz, emtricitabine and tenofovir.

In some embodiments, the subject is being administered a combination comprising emtricitabine, ritonavir and tenofovir. In some embodiments, the combination further includes at least one selected from the group of: lopinavir, atazanavir, fosamprenavir and darunavir. In some embodiments, the subject is being administered a combination comprising emtricitabine, lopinavir, ritonavir and tenofovir. In some embodiments, the subject is being administered a combination comprising atazanavir, emtricitabine, ritonavir and tenofovir. In some embodiments, the subject is being administered a combination comprising fosamprenavir, emtricitabine, ritonavir and tenofovir. In some embodiments, the subject is being administered a combination comprising darunavir, emtricitabine, ritonavir and tenofovir.

In some embodiments, the subject is being administered a combination comprising lamivudine, lopinavir and ritonavir. In some embodiments, the combination further comprises at least one selected from the group of: abacavir and zidovudine. In some embodiments, the subject is being administered a combination comprising lamivudine, abacavir, lopinavir and ritonavir. In some embodiments, the subject is being administered a combination comprising lamivudine, lopinavir, ritonavir and zidovudine.

In some embodiments, the subject is being administered a combination comprising lopinavir and ritonavir. In some embodiments, the combination further comprises at least one selected from the group of: emtricitabine, tenofovir, lamivudine, abacavir, zidovudine and didanosine. In some embodiments, the subject is being administered a combination comprising didanosine, lopinavir, ritonavir and tenofovir. In some embodiments, the subject is being administered a combination comprising emtricitabine, lopinavir, ritonavir and zidovudine. In some embodiments, the subject is being administered a combination comprising lamivudine, lopinavir, ritonavir and zidovudine.

In some embodiments, the subject is being administered a combination comprising lamivudine, efiravenz and zidovudine. In some embodiments, the subject is being administered a combination comprising lamivudine, abacavir, atazanavir and ritonavir. In some embodiments, the subject is being administered a combination comprising emtricitabine, nevirapine and tenofovir. In some embodiments, the subject is being administered a combination comprising lamivudine, nelfinavir and zidovudine. In some embodiments, the subject is being administered a combination comprising lamivudine, abacavir and zidovudine.

Also provided herein is a method of treating HIV-associated or highly active antiretroviral therapy (HAART)-associated diarrhea in an HIV positive subject, wherein the method includes administering a composition comprising crofelemer to the subject, wherein the composition does not significantly inhibit a CYP enzyme in vivo.

Also provided herein is a method of treating HIV-associated or highly active antiretroviral therapy (HAART)-associated diarrhea in an HIV positive subject, wherein the method includes administering a composition comprising crofelemer to the subject, wherein the composition does not significantly inhibit the activity of a drug transporter in vivo.

Also provided herein is a method of treating HIV-associated or highly active antiretroviral therapy (HAART)-associated diarrhea in an HIV positive subject, wherein the method includes administering a composition comprising crofelemer to the subject, wherein the composition does not cause deterioration of immune status in the subject.

In some embodiments, provided herein is a method of treating HIV-associated or highly active antiretroviral therapy (HAART)-associated diarrhea in an HIV positive subject, wherein the method includes administering a composition comprising crofelemer to the subject, wherein the composition has limited systemic exposure in vivo.

Also provided herein is a method of treating HIV-associated or highly active antiretroviral therapy (HAART)-associated diarrhea in an HIV positive subject, wherein the method includes administering a composition comprising crofelemer to the subject, wherein the composition has negligible permeability as measured by in vitro permeability assays.

Also provided herein is a method of treating HIV-associated or highly active antiretroviral therapy (HAART)-associated diarrhea in an HIV positive subject, wherein the method includes administering a composition comprising crofelemer to the subject, wherein the composition does not provoke a significant adverse event in vivo. In some embodiments, the adverse event is at least one selected from the group of: dyspepsia, flatulence, abdominal pain, hemorrhoids, upper respiratory tract infection, and urinary tract infection.

In some embodiments, treatment includes, for example, administering crofelemer, or a composition comprising the same, to the subject at a dosage of about 250 mg to about 1000 mg per day; 250 mg per day; 1000 mg per day; about 125 mg two times per day; or about 500 mg two times per day of crofelemer.

In some embodiments, the subject is administered crofelemer, or a composition comprising the same, at a dosage of 125 mg crofelemer BID.

In some embodiments, the subject is administered crofelemer, or a composition comprising the same, for about 8 days, about 2, 4, 6, 8, 10, 12, 14, 16, 18, 20, 22, 24 or 26 or more weeks. In some embodiments, crofelemer, or a composition comprising the same, is administered to the subject for between about one month and about six months. In some embodiments, crofelemer, or a composition comprising the same, is administered to the subject for between about three days and about six months. In some embodiments, crofelemer, or a composition comprising the same, is administered to the subject for at least about eight days. In some embodiments, crofelemer, or a composition comprising the same, is administered to the subject for between about eight days and about 24 weeks. In some embodiments, crofelemer, or a composition comprising the same, is administered to the subject for about six months. In some embodiments, crofelemer, or a composition comprising the same, is administered to the subject for at least about six months. In some embodiments, crofelemer, or a composition comprising the same, is administered to the subject for the duration of the subject's HIV infection. In other embodiments, the composition is administered to the subject for the remainder of the subjects life. Length of treatment or administration may vary depending on the type and length of disease and the proper length of treatment may be easily determined by one of skill in the art having the benefit of this disclosure. Treatment may be prior to infection, during infection, or after suspected infection and for a period of time suggested by a medical professional to reduce or eliminate diarrhea.

The composition comprising crofelemer may be administered, for example, once a day, twice a day, three times a day, or four times or more often as necessary per day. The composition may be administered in dosages of between about 25 mg BID to about 3000 mg TID of crofelemer. In some embodiments, crofelemer is administered from between about 125 mg to about 1000 mg per day. In some embodiments, crofelemer is administered between 125 mg BID to about 500 mg BID, depending on symptoms. In some embodiments, crofelemer is administered as 125 mg BID. In some embodiments, crofelemer is administered as 500 mg BID. Compositions containing crofelemer may be administered orally, for example, in tablet form, powered form, liquid form or in capsules.

In exemplary embodiments, the subject is administered 250, 500, or 1000 mg/day of crofelemer.

In some embodiments, the subject is administered 125, 250 or 500 mg crofelemer p.o. b.i.d (orally twice per day). Other appropriate dosages for methods may be determined by health care professionals or by the subject. The amount of crofelemer administered daily may be increased or decreased based on the weight, age, health, sex or medical condition of the subject. One of skill in the art would be able to determine the proper dose for a subject based on this disclosure and the data presented in the Examples, which follow.

Subjects in need thereof include subjects having or that are susceptible to or who have HIV-associated diarrhea or HAART associated diarrhea.

In some embodiments, crofelemer may be administered in combination with other compounds, including for example, anti-diarrheal agents or anti-HIV agents, including, for example, anti-retroviral agents. In some embodiments, crofelemer may be administered in combination with at least one of: ritanovir, tenofovir, emtricitabine, lamicudine, lopinavir and efavirenz.

Also provided herein are methods of treating HIV-associated diarrhea or highly active antiretroviral therapy (HAART)-associated diarrhea in an HIV positive subject that has previously used protease inhibitors, comprising: administering about 250 mg to about 1000 mg per day; administering about 250 mg per day; administering about 500 mg per day; administering about 1000 mg per day; administering about 125 mg two times per day; administering about 250 mg two times per day; or administering about 500 mg two times per day of crofelemer, or a composition comprising the same, to the subject, wherein administration of crofelemer or a composition comprising the same does not affect, inhibit or interfere with the activity of the protease inhibitor. As used herein, “previously used” includes, for example, subjects who have used protease inhibitors (PIs) prior to crofelemer therapy or whose administration of protease inhibitors is overlapping with that of crofelemer therapy but began prior to the first dose of crofelemer therapy.

### Pharmaceutical Preparations

Also provided herein are pharmaceutical compositions, comprising an effective amount of a crofelemer described herein and a pharmaceutically acceptable carrier. In a further embodiment, the effective amount is effective to treat HIV-associated diarrhea and/or HAART-associated diarrhea.

Examples of the preparation and use of crofelemer have been described in U.S. Pat. No. 7,556,831, US Patent Publication 20070254050 and US Patent Publication 20080031984, each of which is incorporated herein by reference in its entirety.

In some embodiments, a pharmaceutical composition comprising crofelemer and a pharmaceutically acceptable carrier is provided.

The pharmaceutical compositions described herein may further comprise excipients, for example, one or more of a diluting agent, binding agent, lubricating agent, disintegrating agent, coloring agent, flavoring agent or sweetening agent. Compositions may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In certain embodiments, these pharmaceutical compositions are suitable for topical or oral administration to a subject. In other embodiments, as described in detail below, the pharmaceutical compositions may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intrarectally, for example, as a pessary, cream or foam; or (5) aerosol, for example, as an aqueous aerosol, liposomal preparation or solid particles containing the compound.

The phrase “pharmaceutically acceptable” refers to crofelemer as described herein, compositions containing crofelemer, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing crofelemer include those suitable for oral, nasal, topical (including buccal and sublingual), rectal, vaginal, aerosol and/or parenteral administration. The compositions may conveniently be presented in unit dosage form and may be prepared by any methods known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.01% to about 99% of active ingredient, for example, from about 5% to about 70%, or from about 10% to about 30%.

Liquid dosage forms for oral or rectal administration of crofelemer may include, for example, pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to crofelemer may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Dosage forms for the topical or transdermal administration of crofelemer can include, for example, powders, sprays, ointments, pastes, creams, lotions, gels, solutions, patches and inhalants. The ointments, pastes, creams and gels may contain, in addition to crofelemer, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a croflemer, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and non-aqueous carriers which may be employed in the pharmaceutical compositions can include, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In some embodiments, crofelemer is enterically coated so as to protect it from degradation by the acidic conditions of the stomach and/or from interactions with proteins, such as pepsin, present in the stomach, e.g., an enteric protected formulation. In some embodiments, crofelemer is in tablet form. In some embodiments, the tablet is enteric coated, e.g., coated with Eudragit®. In some embodiments, crofelemer is formulated as an enteric coated bead or as granules in an enteric coated capsule shell. In some embodiments, crofelemer is formulated in a delayed release composition.

In some embodiments, the composition is formulated with a compound or compounds which neutralize stomach acid. In some embodiments, the pharmaceutical composition containing crofelemer is administered either concurrent with, subsequent to, or after administration of a pharmaceutical composition which neutralizes stomach acid. Compounds, such as antacids, which are useful for neutralizing stomach acid include, but are not limited to, aluminum carbonate, aluminum hydroxide, bismuth subnitrate, bismuth subsalicylate, calcium carbonate, dihydroxyaluminum sodium carbonate, magaldrate, magnesium carbonate, magnesium hydroxide, magnesium oxide, and mixtures thereof. Compounds that are able to reduce the secretion of stomach acid and/or are able to reduce the acidity of stomach fluid are well known in the art and include, but are not limited to, antacids (aluminum hydroxide, aluminum carbonate, aluminum glycinate, magnesium oxide, magnesium hydroxide, magnesium carbonate, calcium carbonate, sodium bicarbonate), stomach acid blockers and a combination of any of the foregoing. In general, any drug that has been approved for sale by the relevant government agency and is able to reduce the production of stomach acid and/or reduce the acidity of stomach fluid can be administered in combination with an inhibitor molecule, such as crofelemer, in accordance with the methods presented herein.

In embodiments where crofelemer is not enteric coated, crofelemer is formulated with one or more compounds that are able to reduce the secretion of stomach acid and/or able to reduce the acidity of stomach fluid. In an exemplary embodiment, crofelemer is formulated in a controlled release (delayed release) composition, such as Merck GEM, Alza OROS, wax matrix (release is primarily delayed until after the formulation passes out of the stomach and into the intestine).

Also provided herein are pharmaceutical formulations of crofelemer comprising the composition along with a pharmaceutically acceptable vehicle, at a dose which is therapeutically effective at treating HIV-associated diarrhea or HAART-associated diarrhea. In some embodiments, a directly compressible crofelemer (e.g., crofelemer that can be directly compressed, without excipients, into a tablet of pharmaceutically acceptable hardness and friability) is compressed into a tablet, optionally with a lubricant, such as, but not limited to, magnesium stearate, wherein the tablet is enteric coated. These formulations can be prepared by methods known in the art, see, e.g. methods described in Remington's Pharmaceutical Sciences, 18th Ed., ed. Alfonso R. Gennaro, Mack Publishing Co., Easton, Pa., 1990.

In some embodiments, the composition comprises crofelemer (CAS 148465-45-6).

In some embodiments, the composition is enteric coated. Enteric coatings are those coatings that remain intact in the stomach, but will dissolve and release the contents of the dosage form once the composition reaches the small intestine. A large number of enteric coatings are prepared with ingredients that have acidic groups such that, at the very low pH present in the stomach, e.g. at pH 1.5 to 2.5, the acidic groups are not ionized and the coating remains in an undissociated, insoluble form. At higher pH levels, such as in the environment of the intestine, the enteric coating is converted to an ionized form, which can be dissolved to release crofelemer. Other enteric coatings remain intact until they are degraded by enzymes in the small intestine, and others break apart after a defined exposure to moisture, such that the coatings remain intact until after passage into the small intestines.

Polymers which are useful for the preparation of enteric coatings include, but are not limited to, shellac, starch and amylose acetate phthalates, styrene-maleic acid copolymers, cellulose acetate succinate, cellulose acetate phthalate (CAP), polyvinylacetate phthalate (PVAP), hydroxypropylmethylcellulose phthalate (grades HP-50 and HP-55), ethylcellulose, fats, butyl stearate, and methacrylic acid-methacrylic acid ester copolymers with acid ionizable groups. In some embodiments, the pharmaceutical composition contains a polymeric proanthocyanidin composition (e.g. crofelemer), and the enteric coating polymer is Eudragit® L 30D, an anionic copolymer of methacrylic acid and methyl acrylate with a mean molecular weight of 250,000 Daltons. In some embodiments, the enteric coating polymer is Eudragit® L 30D-55. Application of the enteric coating to the crofelemer composition can be accomplished by any method known in the art for applying enteric coatings. For example, but not by way of limitation, the enteric polymers can be applied using organic solvent based solutions containing from 5 to 10% w/w polymer for spray applications and up to 30% w/w polymer for pan coatings. Solvents that are commonly in use include, but are not limited to, acetone, acetone/ethyl acetate mixtures, methylene chloride/methanol mixtures, and tertiary mixtures containing these solvents. Some enteric polymers, such as methacrylic acid-methacrylic acid ester copolymers can be applied using water as a dispersant. The volatility of the solvent system can be tailored to prevent sticking due to tackiness and to prevent high porosity of the coating due to premature spray drying or precipitation of the polymer as the solvent evaporates.

In some embodiments, the pharmaceutical composition comprising crofelemer is formulated as enteric coated granules or powder (e.g. microspheres with a diameter of 300-500 μm) provided in either hard shell gelatin capsules or suspended in an oral solution for pediatric administration. The enteric coated powder or granules may also be mixed with food, particularly for pediatric administration.

The granules and powder can be prepared using any method known in the art, such as but not limited to, crystallization, spray-drying or any method of comminution, for example, using a high speed mixer/granulator. Exemplary formulations may be found, for example, in U.S. Pat. No. 7,341,744, U.S. Patent Publication No. 2007/0254050 and U.S. Patent Publication No. 2009/0148397, each of which is incorporated herein by reference in its entirety.

Regardless of the route of administration selected, crofelemer can be formulated into pharmaceutically-acceptable dosage forms by methods known to those of skill in the art.

In some embodiments, crofelemer, or a composition comprising the same, and a second drug therapy (e.g. a therapy to treat an HIV infection) can be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In some embodiments, crofelemer, or a composition comprising the same, and at least a second drug therapy (e.g. a therapy to treat an HIV infection) can be administered within the same patient's visit.

### Kits

Kits are also provided herein, for example, kits for treating a diarrhea, e.g., HIV-associated diarrhea or HAART-associated diarrhea, in a subject. The kits may contain, for example, crofelemer or a pharmaceutical composition comprising crofelemer and instructions for use. The instructions for use may contain prescribing information, dosage information, storage information, and the like.

Label instructions include, for example, instructions to take the crofelemer for at least 3 days for the treatment of HIV-associated or HAART-associated diarrhea. In some embodiments, the label can include, for example, instructions to take from between 125 mg BID to 500 mg BID of crofelemer until resolution of symptoms or for 24 weeks for treatment or HIV-associated diarrhea. In some embodiments, the label can include, for example, instructions to take 125 mg BID of crofelemer until resolution of symptoms or for 24 weeks for treatment or HIV-associated diarrhea. In some embodiments, the label can include, for example, instructions to take 500 mg BID of crofelemer until resolution of symptoms or for 24 weeks for treatment or HIV-associated diarrhea.

In some embodiments, the label can include, for example, instructions to take the crofelemer from between 125 mg BID to 500 mg BID of crofelemer until resolution of symptoms or for 26 weeks for treatment or HIV-associated diarrhea. In some embodiments, the label can include, for example, instructions to take 125 mg BID of crofelemer until resolution of symptoms or for 26 weeks for treatment or HIV-associated diarrhea. In some embodiments, the label can include, for example, instructions to take 250 mg BID of crofelemer until resolution of symptoms or for 26 weeks for treatment or HIV-associated diarrhea. In some embodiments, the label can include, for example, instructions to take 500 mg BID of crofelemer until resolution of symptoms or for 26 weeks for treatment or HIV-associated diarrhea.

In some embodiments, the label can include, for example, information that administration of crofelemer, or a composition comprising the same, minimally interacts with a therapy to treat an HIV infection. In some embodiments, the label can include information that crofelemer, or a composition comprising the same, has been shown not to significantly affect, inhibit or interfere with the activity of a therapy to treat an HIV infection, such as, for example, at least one selected from the group of: ritanovir, tenofovir, emtricitabine, lamicudine, lopinavir and efavirenz. In some embodiments, the label can include information that crofelemer, or a composition comprising the same, does not significantly inhibit the activity of at least a CYP enzyme. In some embodiments, the label can include information that crofelemer, or a composition comprising the same, does not significantly inhibit the activity of drug transporters. In some embodiments, the label can include information that crofelemer, or a composition comprising the same, does not have significant systemic exposure in patients.

### EXAMPLES

It will be appreciated that the invention should not be construed to be limited to the example, which is now described; rather, the invention is construed to include any and all applications provided herein and all equivalent variations within the skill of the ordinary artisan.

### Example 1

### Pulmonary Effects of Orally Administered Crofelemer in Rats

Three treatment groups of eight male rats were administered Crofelemer at respective dose levels of 60, 200, and 600 mg/kg. An additional group of eight male rats served as control animals and were administered the vehicle, purified water. Crofelemer and vehicle were administered at a dose volume of 10 mL/kg. One additional group of eight male rats received the positive control article, baclofen, at a dose level of 100 mg/kg and a dose volume of 15 mL/kg. Crofelemer, positive control article, and vehicle were administered to all groups via oral gavage.

Observations for mortality, morbidity, injury, and the availability of food and water were conducted at least twice daily for all animals. Clinical observations were conducted prior to dosing, approximately 1 hour postdose, and following completion of the pulmonary monitoring periods (approximately 4 hours postdose). Body weights were measured and recorded prior to dosing (Day 1). Pulmonary function (respiratory rate, tidal volume, and minute volume) was monitored for approximately 1 hour prior to dosing to establish baseline and for approximately 4 hours postdose. Following the pulmonary monitoring periods, all animals were euthanized and the carcasses were discarded without further evaluation.

Crofelemer, administered orally to male rats at doses of 60, 200, and 600 mg/kg did not produce any effects on mortality or any of the quantitative respiratory endpoints over the course of the study. With respect to the basic respiratory endpoints evaluated in this study, oral administration of crofelemer produced no adverse effects in rats at doses up to and including 60 mg/kg.

### Example 2

### 13-Week Oral Toxicity Study of Crofelemer Administered to Mice

Three treatment groups of 15 male and 15 female mice were administered crofelemer at respective dose levels of 40, 400, and 1200 mg/kg/day. One additional group of 15 animals/sex served as the control and received the vehicle, purified water. The vehicle or crofelemer was administered to all groups at a dose volume of 10 mL/kg. Additionally, four groups of eight or 39 animals/sex/group served as toxicokinetic (TK) animals and received the control or crofelemer in the same manner as the main study groups at respective dose levels of 0, 40, 400, or 1200 mg/kg/day. Due to mortalities, the main study and TK animals at 1200 mg/kg/day were administered crofelemer for up to 55 or 56 days, respectively.

Observations for morbidity, mortality, injury, and the availability of food and water were conducted twice daily for all animals. Detailed clinical observations for clinical signs were conducted weekly on all main study animals. Body weights were measured and recorded weekly on all animals. Food consumption was measured and recorded weekly on all main study animals. Ophthalmoscopic examinations were conducted pretest and prior to necropsy for all main study animals. Blood samples for clinical pathology evaluations were collected from main study animals in extremis and at termination. Blood samples for determination of the plasma concentrations of crofelemer were collected from designated TK animals at designated time points on Days 1, 56, and 91. After blood collection, the TK animals were euthanized and the carcasses were discarded, with the exception of designated animals at 1200 mg/kg/day. The toxicokinetic parameters were determined for crofelemer from concentration time data in the test species. At study termination, necropsy examinations were performed and organ weights were recorded for all main study animals and designated TK animals at 1200 mg/kg/day. Tissues were microscopically examined for main study animals at 0, 400, and 1200 mg/kg/day. Beginning on Day 7, a limited gross necropsy examination was performed on any TK animal euthanized in extremis or found dead in an effort to determine the cause of death. Tissues were collected and preserved for possible future examination from main study animals at 40 mg/kg/day and designated TK animals at 1200 mg/kg/day.

Twice-daily oral gavage administration of crofelemer at 0, 40, or 400 mg/kg/day for 13 weeks or 1200 mg/kg/day for 8 weeks in mice was only tolerated in females at 40 mg/kg/day. Crofelemer-related mortalities were evident in a single male at 40 mg/kg/day and in both sexes at 400 and 1200 mg/kg/day. Crofelemer-related body weight effects were evident at ≧400 mg/kg/day in both sexes, and food consumption effects were evident at 40 mg/kg/day in females and ≧400 mg/kg/day in both sexes. Clinical pathology, organ weight, and macroscopic effects were observed in both sexes at 1200 mg/kg/day. Due to mortality at 40 mg/kg/day, there was no No-Observed-Adverse-Effect-Level (NOAEL) in males; however, the NOAEL was determined to be 40 mg/kg/day in females.

### Example 3

### Neurobehavioral Evaluation of Orally Administered Crofelemer in Rats

Three treatment groups of six male rats were administered crofelemer at respective dose levels of 60, 200, and 600 mg/kg. One additional group of six male rats served as the control and received the vehicle, purified water. Another additional group of six male rats received the positive control article, chlorpromazine hydrochloride, at a dose level of 20 mg/kg. Crofelemer, positive control article, or vehicle was administered to all groups once via oral gavage at a dose volume of 10 mL/kg.

Observations for morbidity, mortality, injury, and the availability of food and water were conducted at least twice daily for all animals. Clinical observations were conducted following each functional observational battery (FOB) examination. FOB evaluations were conducted predose and at approximately 1 and 24 hours postdose. Body weights were measured and recorded prior to dosing on Day 1. At study termination, all animals were euthanized and the carcasses were discarded without further evaluation.

Crofelemer administered orally to male rats at doses of 60, 200, and 600 mg/kg did not produce any effects on mortality, clinical observations, body weight, or any of the neurobehavioral measures tested. Therefore, with respect to the basic neurobehavioral endpoints evaluated in this study, oral administration of crofelemer produced no effects in rats at doses up to and including 600 mg/kg.

### Example 4

### Effects of Orally Administered Crofelemer on Gut Motility Function in the Rat

Three treatment groups of eight male rats were administered crofelemer at respective dose levels of 60, 200, and 600 mg/kg. One additional group of eight male rats received the positive control article, morphine, at a dose level of 20 mg/kg. An additional group of eight male rats served as the control and received the vehicle, purified water. The vehicle, positive control article, or crofelemer was administered to all groups via oral gavage once on Day 1 of the study at a dose volume of 10 mL/kg. Approximately 1 hour after administration, the test meal, 5% charcoal suspension in 10% Acacia in deionized water, was administered to all animals via oral gavage at a dose volume of 10 mL/kg.

Observations for morbidity, mortality, injury, and the availability of food (except during fasting periods) and water were conducted at least twice daily for all animals. Clinical observations were conducted prior to dosing, and prior to termination. Body weights were measured and recorded prior to dosing on Day 1. Approximately 30 minutes following test meal administration all animals were euthanized, the small intestine was surgically removed, and the total intestine length and the distance the charcoal traveled were both measured. The carcasses were discarded without further evaluation.

Crofelemer, administered orally to male rats at doses of 60, 200, or 600 mg/kg did not produce mortality or any clinical observations. Crofelemer-related, dose-dependent decreases in gastrointestinal propulsion were noted in all crofelemer-treated groups; however, statistically significant decreases were only noted following the 200 and 600 mg/kg doses. Due to low recovery values, the dose levels actually administered to the animals in the 60 and 200 mg/kg groups were 51 and 169 mg/kg, respectively.

### Example 5

### Cardiovascular Effects of Orally Administered Crofelemer in the Beagle Dog

The same four male beagle dogs were administered the control article, placebo tablets in gelatin number 12 Torpac lock ring gelatin capsules (0 mg/kg), and crofelemer at dose levels of approximately 60, 200, and 600 mg/kg according to a modified Latin square design, with one animal/sex/treatment dosing each week followed by at least a 7 day washout period between administrations, until each animal received all treatments. The control article and crofelemer were administered to all animals orally via gelatin capsule.

The animals were previously surgically instrumented with radio transmitters for measurement of body temperature, blood pressure, heart rate, and the electrocardiogram (ECG). Body temperature, systolic, diastolic, and mean arterial blood pressures, heart rate, and ECG parameters (QRS duration and the RR, PR, and QT intervals) were monitored continuously from at least 2 hours prior to dosing until at least 20 hours postdose. Nine days prior to the first administration, untreated animals were continuously monitored for cardiovascular endpoints for at least 22 hours. These data were used in the calculation of the corrected QT interval throughout the study.

Observations for morbidity, mortality, injury, and the availability of food and water were conducted at least twice daily for all animals. Clinical observations were conducted prior to dosing and following completion of the cardiovascular monitoring period. Body weights were measured and recorded on the day prior to each administration. At study termination, the animals were transferred to the stock colony.

Crofelemer administered orally to male dogs at doses of 60, 200, and 600 mg/kg did not produce mortality nor any effects on the ECG over the course of the study. Following doses of 200 and 600 mg/kg the test article produced clinical observations of red, black, or brown feces; soft, watery, and/or mucoid feces; and/or black or brown material below the cage (fecal material). Therefore, with respect to all the physiological parameters evaluated as a part of this cardiovascular study, a no-observable-adverse-effect-level (NOAEL) of 600 mg/kg has been established.

### Example 6

### Effects of Crofelemer on Herg K+Currents in Hek-293 Cells

The hERG ion channel was stably expressed in a subclone (HEK-293/hERG) of the HEK-293 cell line. The effect of crofelemer was measured on the maximum amplitude of the tail current. This parameter was determined from current traces obtained from voltage-clamped HEK-293/hERG cells, using patch-clamp techniques in the whole cell configuration.

Crofelemer was tested at 5 concentrations for the first set of experiments: 0.001 μM, 0.01 μM, 0.1 μM, 1.0 μM, and 10.0 μM and 6 concentrations for the second set of experiments: 0.1 μM, 0.3 μM, 1 μM, 3 μM, 10 μM, and 30 μM. Positive control article was tested at 10 μM. Negative control article was deionized water.

Crofelemer inhibited hERG tail current in a dose-dependent manner. The estimated IC50 values were 1.79 μM for the first set of experiments and 1.75 μM for the second set of experiments. Cisapride (positive control, 10 μM) inhibited hERG tail current by an average of 99.67% and 100.47% for the first and second sets of experiments respectively, which is consistent with its known pharmacological action.

### Example 7

### Effects of Food on the PK of Crofelemer 500 Mg

A total of 28 subjects were to be enrolled into this study. Subjects were randomized on Day 1 at a 1:1 ratio to Group 1 (fasted then fed) or Group 2 (fed then fasted). Randomization was stratified by sex. Each subject received a single dose of crofelemer 500 mg (administered orally as 2×250 mg tablets) with a high fat meal (crofelemer fed) and after fasting (crofelemer fasted). The fasted/fed study periods were separated by 7 days. The sequence of fasted/fed or fed/fasted dosing on Days 1 and 8 were determined by randomization on Day 1.

Blood samples for PK analyses of crofelemer were collected pre-dose and up to 48 hours post-dose following both fasted and fed single-dose treatment administrations.

During the food effect study period, subjects fasted overnight (no food for approximately 9.5 hours) prior to administration of a high fat breakfast (crofelemer fed) or fasted overnight for 10 hours prior to administration of the single dose of study drug (crofelemer fasted).

Assessments of the treatment regimen on the relative bioavailability of crofelemer were based on comparisons of plasma concentrations of crofelemer across each study phase. Whole blood samples were drawn at the following times (actual blood collection times were recorded in the source/eCRF): Days 1 and 8: pre-dose (approximately 1 hour before dosing), and at 0.5, 1, 2, 4, 6, 8, 10, 12, 16, 24, 30, 36, 42, and 48 hours post-dose.

A pharmacokinetic/pharmacodynamic (PK/PD) analysis was intended to be performed using all subjects who had paired ECG and plasma concentrations for crofelemer. The PK/PD pharmacokinetic-pharmacodynamic analysis was not done due to insufficient pharmacokinetic PK data.

This analysis was also designed to assess the relationship between the concentration of crofelemer and QTcF. However, only 3 samples were found to have concentrations above the LLOQ of 50 ng/mL so the relationship of crofelemer concentration to QTcF could not be assessed.

The mean baseline corrected change in heart rate showed a decrease in heart rate of −1.1 bpm and −1.0 bpm for crofelemer fasting and fed, respectively, at 4 hours. The mean baseline corrected change in heart rate showed an increase in heart rate of 3.5 bpm and 1.6 bpm for crofelemer fasting and fed, respectively, at 12 hours. The heart rate changes were of no clinical significance. There were no tachycardic or bradycardic outliers on crofelemer fasting or fed.

### Example 8

### Efficacy and Safety of Crofelemer for the Treatment of HIV Associated Diarrhea

This study was conducted to determine if treatment with crofelemer 125 mg, 250 mg and 500 mg orally (p.o.) twice daily improves the frequency, consistency, and urgency of bowel movements in subjects with HIV-associated diarrhea compared to placebo. Male or female subjects aged ≧18 years, with HIV-1 infection confirmed by standard serological tests and/or viral load and history of diarrhea of at least 1 month duration

The study was a randomized, double-blind, parallel-group, placebo-controlled, multicenter study. The study was done in two stages. Both stages consisted of a 10+4 day, single-blind, placebo screening phase; followed by randomization and a 31-day, double-blind, placebo-controlled treatment phase; and concluded with a 20-week placebo-free extension phase. In Stage I (dose selection stage), the double-blind phase had four arms: three doses of crofelemer (125 mg b.i.d., 250 mg b.i.d., and 500 mg b.i.d.) and placebo b.i.d. The chance of receiving crofelemer or placebo was 3:1 (1:1:1:1 ratio). Subjects on crofelemer who were rolled over into the extension phase remained on their same dose; if on placebo, the subject was re-randomized in the extension phase to one of the three aforementioned doses. After Stage I was completed, an interim analysis was conducted, and the dose of crofelemer that appeared to be more effective, safer, and/or better tolerated was selected to be used exclusively in Stage II. Stage II was designed to have only two arms in the double blind, placebo-controlled treatment phase: the selected dose of crofelemer (crofelemer 125 mg b.i.d.) and placebo. Accordingly, the chances of receiving crofelemer or placebo in Stage II is 1:1. All subjects rolling over into the 20-week placebo-free extension phase in Stage II were assigned the previously selected dose of crofelemer. During both Stage I and Stage II, subjects were first entered into a single-blind placebo screening phase lasting 10±4 days during which time bowel movement frequency, consistency, and urgency was measured. Antiretroviral therapy and therapy for associated conditions (including prophylactic antibiotics for Pneumocystis carinii (PCP) or infection must have remained at a constant level from four weeks prior to screening through the placebo-controlled treatment phase. Any changes in antiretroviral therapy during any point in the study were reported to the site and documented on the subject's case report form.

The key efficacy analyses were based on the data from the 4-week efficacy assessment period of the placebo-controlled treatment phase. Analysis of an endpoint was based on the Intent to Treat (ITT) population and compared the proportion of responders in the placebo group to the proportion of responders in the crofelemer 125 mg b.i.d. group. An efficacy endpoint was clinical response, defined as two or less watery bowel movements per week, during at least two of the four weeks of the 4-week efficacy assessment period in the ITT population. The secondary efficacy variables during the 4-week efficacy assessment period in the ITT population were:
The number of bowel movements per day
The number of watery bowel movements per day;
The score for daily abdominal pain or discomfort;
The score for daily stool consistency;
The number of days per week that subjects experienced urgency;
The number of days per week that subjects experienced fecal incontinence; and
Proportion of subjects undergoing an unscheduled visit for a significant worsening or clinically significant exacerbation of diarrhea during the 4-week efficacy assessment period.

This study consisted of a dose-selection stage, an interim analysis period, and a dose-assessment stage.

### Stage I: Dose-Selection Stage

Subjects were randomized 1:1:1:1, at approximately 50 subjects per treatment group: crofelemer 125 mg p.o. b.i.d.; crofelemer 250 mg p.o. b.i.d.; crofelemer 500 mg p.o. b.i.d.; and placebo p.o. b.i.d.

Crofelemer 125 mg, 250 mg, and 500 mg, or matching placebo, was administered as a tablet combination orally twice daily with fluids at least one half hour before the morning and evening meals.

The double-blind, placebo-controlled treatment phase consisted of an initial 3-day run-in period (Days −3 to −1) followed by a 4-week efficacy assessment period (Days 1 to 28). The run-in period assured that the effects of study medication were established before the 4-week efficacy assessment period is commenced.

Subjects who completed the placebo-controlled treatment phase entered a 20-week placebo-free extension phase. Subjects in the crofelemer 125 mg p.o. b.i.d., crofelemer 250 mg p.o. b.i.d or crofelemer 500 mg b.i.d. groups continued to receive these therapies throughout the placebo-free treatment phase; subjects who received placebo were re-randomized to receive either crofelemer 125 mg p.o. b.i.d, crofelemer 250 mg p.o. b.i.d. or crofelemer 500 mg p.o. b.i.d (1:1:1). There was no risk of placebo during the 20-week extension phase, and subjects were permitted ad libitum (prn) use of anti-diarrhea medication (ADM).

Stage I ended when about 50 subjects were randomized to each of the four treatment groups. Enrollment was stopped at approximately 50 subjects per treatment group until the interim analysis and decision for Stage II were completed.

Other objectives were to evaluate the effects of crofelemer 125 mg p.o. b.i.d. vs. placebo on:
i. Number of bowel movements per day (frequency)
ii. Number of watery bowel movements per day
iii. Symptom frequency (urgency, fecal incontinence)
iv. Symptom severity (abdominal pain or discomfort); and
v. Daily stool consistency score.

The ratio of randomization to crofelemer 125 mg p.o. b.i.d or placebo p.o. b.i.d was 1:1. Subjects who completed the double-blind treatment phase participated in the 20-week, placebo-free extension phase and received crofelemer 125 mg p.o. b.i.d. Subjects who enrolled in Stage I, and who received either crofelemer 125 mg p.o. b.i.d., 250 mg p.o. b.i.d. or 500 mg p.o. b.i.d, remained on their previously assigned dose. However, subjects were re-assigned to crofelemer 125 mg p.o. b.i.d. if, in the opinion of the investigator, the response to or tolerance to their current dose was inadequate. Treatment remained blinded during this treatment period, including the possibility that subjects were switched to the same dose they had initially been taking.

All study procedures performed in Stage I were otherwise identical to Stage II.

Subjects randomized during Stages I and II were combined and included in sample size calculations and analysis of efficacy and safety.

The following criteria were used for collecting data to evaluate the efficacy of crofelemer on HIV-associated diarrhea.

### Study Diary (IVRS) Definitions

Diarrhea, includes, frequent loose or watery bowel movements

Bowel movement is defined as a trip to the bathroom with evacuation of stool; number of bowel movements means number of trips to the bathroom with evacuation of stool.
Watery bowel movement is defined as stool that can be poured;
Loose bowel movement is defined as soft blobs with no shape or form;
Formed bowel movement is defined as a stool like a soft sausage;
Hard bowel movement is defined as a stool like a hard or lumpy sausage; and
Very hard bowel movement is defined as hard lumps or nuts that are hard to pass.

Urgency is defined as having to rush to the bathroom for a bowel movement

Fecal incontinence is defined as leaking or passing stool at unwanted times (two teaspoons or more of stool).

Abdominal pain or discomfort is defined as pain, cramping, or bloating that is uncomfortable and/or interrupts normal activities.

### Stool Samples

Each sample collected was analyzed as follows:

### Visit 0

Clostridium difficile toxin;
Enteric pathogens, O&P examination;
Giardia-specific antigen by EIA;
Modified acid-fast stain for Cryptosporidium, Cyclospora, and Isospora;
Lactoferrin (qualitative); and
Occult blood.

### Visit 3

Clostridium difficile toxin;
Enteric pathogens, O&P examination;
Giardia-specific antigen by EIA; and
Modified acid-fast stain for Cryptosporidium, Cyclospora, and Isospora.

### Visits 4, 5, 6, 7 and 8

Clostridium difficile toxin;
Enteric pathogens, O&P examination
Giardia-specific antigen by EIA
Modified acid-fast stain for Cryptosporidium, Cyclospora, and Isospora

### Analysis of the Primary Efficacy Variable

An efficacy endpoint is clinical response; subjects are classified responders if they reported two or less watery bowel movements per week, during at least two of the four weeks of the efficacy assessment period of the placebo-controlled treatment phase.

### Analysis of Secondary Efficacy Variables

For every subject, a mean baseline, a mean for Weeks 1 to 4, and change from baseline will be calculated for the following variables:
Number of bowel movements per day;
Number of watery bowel movements per day;
Daily abdominal pain or discomfort score;
Daily stool consistency score;
The number of days per week that subjects experienced urgency; and
The number of days per week that subjects experienced fecal incontinence.

Each of the secondary continuous variables will be analyzed as percent change from baseline.

The daily abdominal pain or discomfort score were assigned scores as follows: none=0, mild=1, moderate=2, severe=3, that is the greater the score the worse the pain or discomfort. Stool consistencies will be assigned scores as follows: 1=very hard, 2=hard, 3=formed, 4=loose, 5=watery for each bowel movement.

The stool consistency score was computed from the mean of these scores each day.

Table 1 below shows the baseline characteristics for the placebo-controlled treatment phase, including the diagnosed cause of diarrhea, the CD4 cell count and the CD4 cell category. This table demonstrates that the subjects in each group were similar.

TABLE 1
Baseline Characteristics in the Placebo-Controlled Treatment Phase

| | | Crofelemer | Crofelemer | Crofelemer | |
|---|---|---|---|---|---|
| Baseline Disease | Placebo | 125 mg * | 250 mg | 500 mg | All Crofelemer |
| Characteristics | (N = 138) | (N = 138) | (N = 54) | (N = 47) | (N = 239) |
| Cause of diarrhea | | | | | |
| Antiretroviral therapy | 104 (75.4%) | 104 (75.4%) | 37 (68.5%) | 30 (63.8%) | 171 (71.5%) |
| HIV infection of intestine | 33 (23.9%) | 32 (23.2% | 15 (27.8%) | 15 (31.9%) | 62 (25.9%) |
| Other | 1 (0.7%) | 2 (1.4%) | 2 (3.7%) | 2 (4.3%) | 6 (2.5%) |
| CD4 cell counts | | | | | |
| N | 138 | 137 | 54 | 46 | 237 |
| Mean | 530.5 | 497.8 | 425.2 | 481.7 | 478.1 |
| SD | 244.79 | 230.88 | 226.13 | 275.18 | 239.81 |
| Median | 518.5 | 479.0 | 374.0 | 421.5 | 429.0 |
| Min | 76 | 111 | 100 | 149 | 100 |
| Max | 1298 | 1183 | 1095 | 1734 | 1734 |
| CD4 cell Category | | | | | |
| <404 | 39 (28.3%) | 55 (39.9%) | 29 (53.7%) | 21 (44.7%) | 105 (3.9%) |
| >=404 | 99 (71.7%) | 32 (59.4%) | 25 (46.3%) | 25 (53.2%) | 132 (55.2%; |

[1] Baseline was the average of daily data from the 7 days prior to first dose day of randomized study drug.
[2] Baseline was the average of daily stool consistency scores from the 7 days prior to first dose day of randomized study drug. The daily score = (1 * # of very hard stools + 2 * # of hard stools + 3 * # of formed stools + 4 * # of loose stools + 5 * # of watery stools)/(# of total stools).
[3] Baseline was the average of daily scores from the 7 days prior to first dose day of randomized study drug, none = 0, mild = 1, moderate = 2, severe = 3.
[4] Baseline = 7 * A/B, A = # of days with event during the 7 days prior to first dose day of randomized study drug, B = # of days with non-missing assessments.

Table 2 below shows additional baseline characteristics of the placebo-controlled treatment phase, including use of antibiotics during the study.

TABLE 2
Baseline Characteristics of the Placebo-Controlled Treatment Phase

| | | | | | |
|---|---|---|---|---|---|
| | | Crofelemer | Crofelemer | Crofelemer | |
| Baseline Disease | Placebo | 125 mg* | 250 mg | 500 mg | All Crofelemer |
| Characteristics | (N = 138) | (N = 138) | (N = 54) | (N = 47) | (N = 239) |

Use of a new antibiotic regimen during placebo-controlled treatment phase

| | | | | | |
|---|---|---|---|---|---|
| Yes | 14 (10.1%) | 9 (6.5%) | 2 (3.7%) | 1 (2.1%) | 12 (5.0%) |
| No | 124 (89.9%) | 129 (92.5%) | 52 (96.3%) | 46 (97.9%) | 227 (95.0%) |

Tables 3 and 3a below show the percentage of subjects with clinical response, e.g., improvement of watery diarrhea, in the placebo-controlled treatment phase, and the change in response from baseline as a function of time, respectively. As can be seen from Table 3, all three treatment groups from Stage I were statistically significant for treatment of watery diarrhea, as well at the combined group of subjects dosed 125 mg in both stages. Table 3a sets forth data indicating that, regardless of treatment group, an endpoint (Clinical Response) demonstrated responsiveness by consistently correlating with other daily assessments collected in the study for changes in symptoms scoring. Responders, i.e., subjects with ≦2 watery stools per week, had significantly greater improvements in daily symptom severity scores than non-responders at each week during the course of the study.

TABLE 3
Percentage of Subjects with Clinical Response in the Placebo-Controlled
Treatment Phase

| | | | |
|---|---|---|---|
| | Crofelemer | Crofelemer | Crofelemer |
| Placebo | 125 mg* | 250 mg | 500 mg |
| (N = 138) | (N = 136) | (N = 54) | (N = 46) |

Stage I

| | | | | |
|---|---|---|---|---|
| Responder - n/Ni (%) | 1/50 (2.0%) | 9/44 (20.5%) | 5/54 (9.3%) | 9/46 (19.6%) |
| Treatment Difference (vs. | | 18.5% | 7.3% | 17.6% |
| Placebo) | | | | |
| 1-Sided 97.5% CI (1) | | (6.0%, ⁰⁰) | (−1.7%, ⁰⁰) | (5.3%, ⁰⁰) |
| 1-Sided P-value (vs. Placebo) [1] | | 0.0019 | 0.0563 | 0.0024 |

Combined

| | | |
|---|---|---|
| Responder - n/Ni (%) | 11/138 | 24/136 |
| | (8.0%) | (17.6%) |
| Treatment Difference (vs. | | 9.6% |
| Placebo) | | |
| 1-Sided 97.5% CI (1) | | (1.2%, ⁰⁰) |
| 1-Sided P-value (vs. Placebo) [1] | | 0.0096 |

TABLE 3a
Responsiveness of an Endpoint (Clinical Response)

| | | | | |
|---|---|---|---|---|
| | Clinical | | | |
| | Response^{a}: | | | |
| | Weekly | Clinical Response^{a}: | | |
| | Responder | Weekly Non- | | |
| | Mean Change | Responder | Difference | |
| Week | from Baseline | Mean Change from | Responder − | |
| Daily Question | (± SD) | Baseline (± SD) | Non-Responder | p-value |

Week 1

| | | | | |
|---|---|---|---|---|
| Daily Watery Stools | −1.75 (0.901) | −0.51 (1.296) | −1.24 | <0.0001 |
| Daily Stool Consistency^{b} | −1.08 (0.589) | −0.19 (0.381) | −0.90 | <0.0001 |
| Daily Abdominal Pain^{c} | −0.41 (0.651) | −0.11 (0.493) | −0.30 | 0.0217 |
| Urgency^{d} | −2.97 (2.299) | −0.75 (1.908) | −2.22 | <0.0001 |
| Fecal Incontinence^{e} | −1.48 (2.007) | −0.43 (1.867) | −1.06 | 0.0144 |
| Daily Stool Frequency | −1.13 (1.299) | −0.31 (1.810) | −0.83 | <0.0001 |

Week 2

| | | | | |
|---|---|---|---|---|
| Daily Watery Stools | −2.03 (1.184) | −0.53 (1.392) | −1.50 | <0.0001 |
| Daily Stool Consistency^{b} | −1.14 (0.672) | −0.17 (0.379) | −0.97 | <0.0001 |
| Daily Abdominal Pain^{c} | −0.61 (0.701) | −0.12 (0.494) | −0.49 | <0.0001 |
| Urgency^{d} | −2.79 (2.418) | −0.79 (2.014) | −2.00 | <0.0001 |
| Fecal Incontinence^{e} | −1.71 (2.071) | −0.44 (1.982) | −1.27 | <0.0001 |
| Daily Stool Frequency | −1.18 (1.331) | −0.34 (1.851) | −0.83 | <0.0001 |

Week 3

| | | | | |
|---|---|---|---|---|
| Daily Watery Stools | −2.00 (1.280) | −0.65 (1.408) | −1.35 | <0.0001 |
| Daily Stool Consistency^{b} | −1.20 (0.734) | −0.22 (0.382) | −0.98 | <0.0001 |
| Daily Abdominal Pain^{c} | −0.51 (0.668) | −0.17 (0.517) | −0.34 | 0.0016 |
| Urgency^{d} | −3.41 (2.215) | −0.98 (2.253) | −2.43 | <0.0001 |
| Fecal Incontinence^{e} | −1.65 (2.228) | −0.64 (2.121) | −1.01 | 0.0015 |
| Daily Stool Frequency | −1.20 (1.314) | −0.41 (1.992) | −0.79 | <0.0001 |

Week 4

| | | | | |
|---|---|---|---|---|
| Daily Watery Stools | −1.89 (1.058) | −0.68 (1.430) | −1.21 | <0.0001 |
| Daily Stool Consistency^{b} | −1.07 (0.645) | −0.24 (0.410) | −0.83 | <0.0001 |
| Daily Abdominal Pain^{c} | −0.48 (0.658) | −0.19 (0.533) | −0.28 | 0.0058 |
| Urgency^{d} | −3.11 (2.411) | −0.99 (2.166) | −2.12 | <0.0001 |
| Fecal Incontinence^{e} | −1.75 (2.140) | −0.57 (2.085) | −1.18 | <0.0001 |
| Daily Stool Frequency | −1.07 (1.086) | −0.43 (2.039) | −0.64 | <0.0001 |

^{a}Clinical response in a week was defined as ≦2 watery stools during a given week.
^{b}Stool consistency response was defined as <4 daily stool consistency score during a given week
^{c}Abdominal pain and discomfort score: 0 = none, 1 = mild, 2 = moderate, and 3 = severe.
^{d}Number of days per week with urgency = 7 * A/B, where A = # of days with urgency in the week, and B = # of days with assessments in the week.
^{e}Number of days per week with fecal incontinence = 7 * A/B, where A = # of days with fecal incontinence in the week, and B = # of days with assessments in the week.
^{f}P-values were obtained from a Wilcoxon rank-sum test for comparison of the responder vs. non-responder groups.

Table 4 below shows the number of weeks of clinical response of subjects in the study. As shown below, the subjects dosed with 500 mg BID had more weeks of response to the treatment.

TABLE 4
Number of Weeks with Clinical Response in the Placebo-Controlled
Treatment Phase

| | | | | |
|---|---|---|---|---|
| Number of | | Crofelemer | Crofelemer | Crofelemer |
| Weeks | Placebo | 125 mg* | 250 mg | 500 mg |
| Responded | (N = 138) | (N = 136) | (N = 54) | (N = 46) |

Stage I

| | | | | |
|---|---|---|---|---|
| Number of | N = 50 | N = 44 | N = 54 | N = 46 |
| Subjects | | | | |
| 0 week | 43 (86.0%) | 32 (72.7%) | 41 (75.9%) | 25 (60.9%) |
| 1 week | 6 (12.0%) | 2 (4.5%) | 8 (14.8%) | 9 (19.6%) |
| 2 weeks | | 2 (4.5%) | 2 (3.7%) | 4 (8.7%) |
| 3 weeks | | 4 (9.1%) | 2 (3.7%) | 4 (8.7%) |
| 4 weeks | 1 (2.0%) | 3 (6.8%) | 1 (1.9%) | 1 (2.2%) |

Crofelemer vs. Placebo [1]

| | | | |
|---|---|---|---|
| Odds Ratio | 2.41 | 2.02 | 4.21 |
| 95% CI | (0.84, 6.87) | (0.74, 5.56) | (1.56, 11.33) |
| p-value | 0.1011 | 0.1727 | 0.0045 |

Table 5 below shows subjects with clinical response by month. As can be seen from the Table, responding subjects and response rate increases the longer a subject was administered crofelemer.

TABLE 5
Subjects with Monthly Clinical Response by Month in the Placebo-Free
Extension Phase

| | Crofelemer | Crofelemer | Crofelemer | |
|---|---|---|---|---|
| | 125 mg* | 250 mg | 500 mg | All Crofelemer |
| Responders in Placebo- | (N = 219) | (N = 68) | (N = 50) | (N = 337) |
| free Extension Phase | n (%) | n (%) | n (%) | n (%) |
| Month 1 | 87/218 (39.9%) | 19/68 (27.9%) | 14/50 (28.0%) | 120/336 (35.7%) |
| Month 2 | 99/208 (47.6%) | 31/60 (51.7%) | 19/48 (39.6%) | 149/316 (47.2%) |
| Month 3 | 111/198 (56.1%) | 30/58 (51.7%) | 17/47 (36.2%) | 158/303 (52.1%) |
| Month 4 | 99/178 (55.6%) | 27/54 (50.0%) | 20/44 (45.5%) | 146/276 (52.9%) |
| Month 5 | 90/155 (58.1%) | 30/53 (56.6%) | 17/42 (40.5%) | 137/250 (54.8%) |

Table 6 below shows the percent of subjects with stool consistency response to crofelemer. As can be seen from Table 6, subjects administered 500 mg and 250 mg BID responded better than did the 125 mg BID or the 250 mg BID.

TABLE 6
Percentage of Subjects with Stool Consistency Response in the Placebo-
Controlled Treatment Phase

| | | | |
|---|---|---|---|
| | Crofelemer | Crofelemer | Crofelemer |
| Placebo | 125 mg* | 250 mg | 500 mg |
| (N = 138) | (N = 136) | (N = 54) | (N = 46) |

Stage I

| | | | | |
|---|---|---|---|---|
| Responder - n/Ni (%) | 11/50 (22.0%) | 14/44 (31.8%) | 20/54 (37.0%) | 23/46 (50.0%) |
| Treatment Difference vs. | | 9.8% | 15.0% | 28.0% |
| Placebo | | | | |
| 1-Sided 97.5% CI [1] | | (−8.1%, ⁰⁰) | (−2.6%, ⁰⁰) | (−8.8%, ⁰⁰) |
| 1-Sided P-value (vs. | | 0.1412 | 0.0470 | 0.0021 |
| Placebo) [1] | | | | |

Combined

| | | |
|---|---|---|
| Responder - n/Ni (%) | 49/186 | 53/136 |
| | (35.5%) | (39.0%) |
| Treatment Difference vs. | | 3.5% |
| Placebo | | |
| 1-Sided 97.5% CI [1] | | [−5.0%, ⁰⁰) |
| 1-Sided P-value (vs. | | 0.1428 |
| Placebo) [1] | | |

Table 7 below shows the number of months of clinical response by subjects on crofelemer in the study. Table 7 again demonstrates that the longer the treatment, the better the response to treatment.

TABLE 7
Number of Months with Clinical Response in the Placebo-Free
Extension Phase

| | Crofelemer | Crofelemer | Crofelemer |
|---|---|---|---|
| Number of Months | 125 mg* | 250 mg | 500 mg |
| Responded | (N = 219) | (N = 66) | (N = 50) |
| 0 month | 69 (31.5%) | 25 (36.8%) | 25 (50.0%) |
| 1 month | 31 (14.2%) | 6 (8.8%) | 4 (8.0%) |
| 2 months | 19 (8.7%) | 10 (14.7%) | 4 (8.0%) |
| 3 months | 29 (13.2%) | 7 (10.3%) | 3 (6.0%) |
| 4 months | 25 (11.4%) | 10 (14.7%) | 4 (8.0%) |
| 5 months | 46 (21.0%) | 10 (14.7%) | 10 (20.0%) |

Table 8 below shows the percentage of Caucasian and Hispanic subjects and all other races with clinical response. This table demonstrates that the Caucasian and Hispanic populations receiving crofelemer responded well to treatment.

TABLE 8
Percentage of Subjects with Clinical Response by Race in the Placebo-
Controlled Treatment Phase

| | | | | |
|---|---|---|---|---|
| | | Crofelemer | Crofelemer | Crofelemer |
| | Placebo | 125 mg* | 250 mg | 500 mg |
| Race: White/Hispanic | (N = 83) | (N = 84) | (N = 44) | (N = 38) |

Stage I

| | | | | |
|---|---|---|---|---|
| Responder - n/Ni (%) | 0/36 (0.0%) | 7/31 (22.6%) | 3/44 (6.8%) | 8/38 (21.1%) |
| Treatment Difference vs. | | 22.6% | 6.5% | 21.1% |
| Placebo | | | | |
| [95% CI] | | [7.9%, 37.3%] | [−0.6%, 14.3%] | [8.14%, 34.0%] |
| P-value vs. Placebo [1] | | 0.0030 | 0.2481 | 0.0053 |

Combined

| Responder - n/Ni (%) | 6/83 (7.2%) | 19/84 (22.6%) | | |
|---|---|---|---|---|
| Treatment Difference vs. | | 15.4% | | |
| Placebo | | | | |
| [95% CI] | | [4.9%, 25.9%] | | |
| P-value (vs. Placebo) [1] | | 0.0083 | | |
| | | Crofelemer | Crofelemer | Crofelemer |
| | Placebo | 125 mg* | 250 mg | 500 mg |
| Race: Other | (N = 55) | (N = 52) | (N = 10) | (N = 8) |

Stage I

| | | | | |
|---|---|---|---|---|
| Responder - n/Ni (%) | 1/14 (7.1%) | 2/13 (15.4%) | 2/10 (20.0%) | 1/8 (12.5%) |
| Treatment Difference vs. | | 8.2% | 12.9% | 5.4% |
| Placebo | | | | |
| [95% CI] | | [−15.6%, 32.0%] | [−15.4%, 41.1%] | [−21.2%, 32.0%] |
| P-value vs. Placebo [1] | | 0.5956 | 0.5504 | 1.0000 |

Combined

| | | |
|---|---|---|
| Responder - n/Ni (%) | 5/55 (9.1%) | 5/52 (9.6%) |
| Treatment Difference vs. | | 0.5% |
| Placebo | | |
| [95% CI] | | [−10.5%, 11.6%] |
| P-value (vs. Placebo) [1] | | 1.0000 |

Note:
Clinical response was defined as <=2 watery stools per week during at least 2 of the 4 efficacy assessment weeks.
Note:
Percentage is based on Ni, number of subjects entered the stage or combine.

In another study, 400 subjects with chronic HIV associated diarrhea were treated with crofelemer or placebo for 7 days in an inpatient setting. Crofelemer was given at doses of 250 mg and 500 mg enteric-coated tablets or 500 mg enteric-coated beads four times daily, compared to a matching placebo. Subjects who responded to treatment were continued in a three-week blinded outpatient phase. The difference in decreased stool weight between subjects receiving crofelemer and subjects receiving placebo was not statistically significant by the primary efficacy analysis.

However, reanalysis of these data revealed that approximately 50% of the study population did not have watery diarrhea at study entry. Evaluation of the population with watery diarrhea and urgency at baseline revealed statistically significant improvement in stool frequency and weight (p<0.05) with treatment. Changes in abnormal (watery and loose) stools were even greater, with significant (p<0.015) improvements in abnormal stool weight and frequency observed at Day 7. Approximately 3 days were necessary for the anti-diarrheal effects of crofelemer to stabilize.

Table 9 below shows additional supportive data showing that crofelemer 125 mg, 250 mg and 500 mg (p.o. b.i.d.) were efficacious at treating HIV associated diarrhea.

TABLE 9
Efficacy Endpoint
Percentage of Subjects with Clinical Response*

| | | | |
|---|---|---|---|
| | Crofelemer | Crofelemer | |
| Placebo | 125 mg | 250 mg | Crofelemer 500 mg |
| (N = 138) | (N = 136) | (N = 54) | (N = 46) |

Stage 1

| | | | | |
|---|---|---|---|---|
| Responder | 1/50 (2.0%) | 9/44 (20.5%) | 5/54 (9.3%) | 9/46 (19.6%) |
| Treatment Difference | | 18.50% | 7.30% | 17.60% |

Stage 2

| | | |
|---|---|---|
| Responder | 10/88 (11.4%) | 15/92 (16.3%) |
| Treatment Difference | | 4.90% |

Combined

| | | |
|---|---|---|
| Responder | 11/138 (8.0%) | 24/136 (17.6%) |
| Treatment Difference | | 9.60% |
| 1-Sided 97.5% CI | | [1.2%, ∞) |
| 1-Sided P-value (vs. | | 0.0096 |
| Placebo) | | |

*≦2 watery BM/wk during ≧2 of 4 wks of the 4-wk placebo controlled period

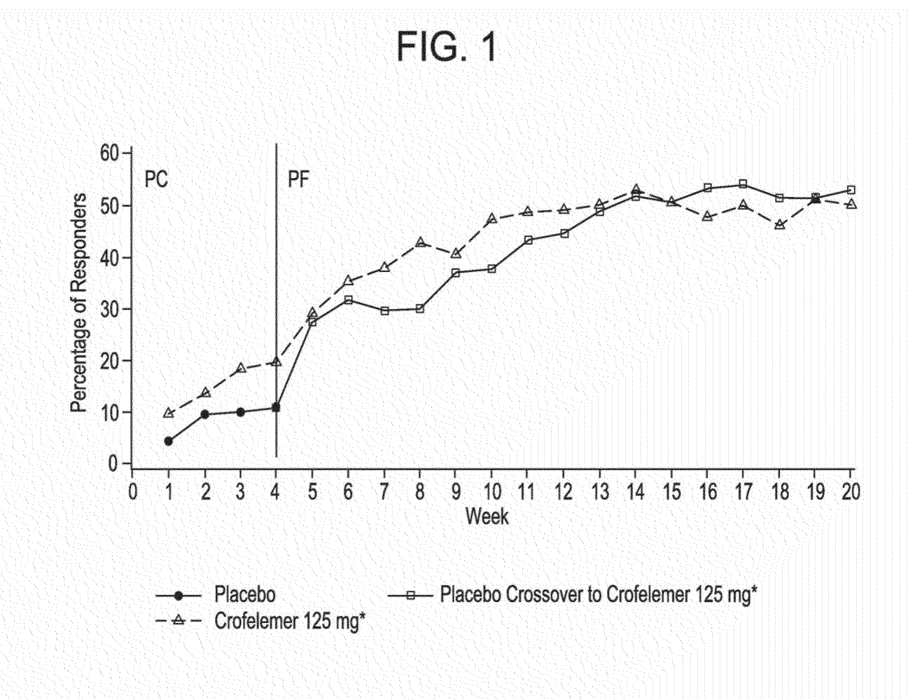

Table 10 below shows that crofelemer is particularly efficacious in treating males with HIV associated diarrhea. Additional subgroup analysis is show in FIG. 1. An endpoint was analyzed in subgroups defined by demographics and baseline characteristics to assess the consistency of the treatment effect. FIG. 1 provides a summary of each of these subgroup analyses, showing the treatment difference in percentage of responders (crofelemer 125 mg BID vs. placebo) with associated confidence intervals and p-values. As shown in the figure, consistent efficacy was observed across subgroups; a higher percentage of subjects treated with crofelemer 125 mg BID experienced clinical response compared with placebo in all subgroups analyzed.

TABLE 10
Effects Across Demographic & Baseline Characteristics - Gender*

| | |
|---|---|
| | Crofelemer 125 mg |
| Placebo (N = 138) | (N = 136) |

Gender: Male

| | | |
|---|---|---|
| Responder - n/Ni (%) | 9/116 (7.8%) | 22/115 (19.1%) |
| Treatment Difference vs | | 11.40% |
| Placebo | | |
| [95% CI] | | [2.7%, 20.1%] |
| P-value vs Placebo | | 0.0124 |

Gender: Female

| | | |
|---|---|---|
| Responder - n/Ni (%) | 2/22 (9.1%) | 2/21 (9.5%) |
| Treatment Difference vs | | 0.40% |
| Placebo | | |
| [95% CI] | | [−16.9%, 17.8%] |
| P-value vs Placebo | | 1 |

*Clinical response was defined as <=2 watery stools per week during at least 2 of the 4 efficacy assessment weeks. Intent-to-Treat Population.

According to one aspect, provided herein are methods of treating HIV associated diarrhea or highly active antiretroviral therapy (HAART) associated diarrhea in a male HIV positive subject, comprising: administering about 250 mg to about 1000 mg per day; administering about 250 mg per day; administering about 500 mg per day; administering about 1000 mg per day; administering about 125 mg two times per day; administering about 250 mg two times per day; or administering about 500 mg two times per day of crofelemer to a male subject in need thereof.

Below, Table 11 shows that subject taking protease inhibitors responded particularly well to treatment with crofelemer.

TABLE 11
Effects Across Demographic & Baseline Characteristics -
Prior use of Protease Inhibitors*

| | |
|---|---|
| Placebo | Crofelemer 125 mg |
| (N = 138) | (N = 136) |

Use of PI at Screening - Yes:

| | | |
|---|---|---|
| Responder - n/Ni (%) | 6/97 (6.2%) | 15/86 (17.4%) |
| Treatment Difference vs Placebo | | 11.30% |
| [95% CI] | | [1.9%, 20.6%] |
| P-value vs Placebo | | 0.0204 |

Use of PI at Screening - No:

| | | |
|---|---|---|
| Responder - n/Ni (%) | 5/41 (12.2%) | 9/50 (18.0%) |
| Treatment Difference vs Placebo | | 5.80% |
| [95% CI] | | [−8.8%, 20.4%] |
| P-value vs Placebo | | 0.5638 |

*Clinical response was defined as <=2 watery stools per week during at least 2 of the 4 efficacy assessment weeks. Intent-to-Treat Population

Accordingly, provided herein are methods of treating HIV-associated diarrhea or highly active antiretroviral therapy (HAART)-associated diarrhea in an HIV positive subject that has previously used protease inhibitors, comprising: administering about 250 mg to about 1000 mg per day; administering about 250 mg per day; administering about 500 mg per day; administering about 1000 mg per day; administering about 125 mg two times per day; administering about 250 mg two times per day; or administering about 500 mg two times per day of crofelemer to a male subject in need thereof, wherein administration of crofelemer or a composition comprising the same does not affect, inhibit or interfere with the activity of the protease inhibitor. As used herein, “previously used” includes, for example, subjects who have used protease inhibitors (PIs) prior to crofelemer therapy or overlapping with crofelemer therapy, but the PI use began prior to the first dose of crofelemer therapy.

TABLE 12
Efficacy Endpoints

Intent-to-Treat
Population

| | |
|---|---|
| Watery BMs/day | No treatment differences between 125 mg vs. Pbo |
| | Stage 1: numerically greater in 500 mg vs. Pbo |
| | (−0.91/day vs. −0.62/day; p = 0.0713) |
| Stool consistency | Significant improvement for 125 mg vs. |
| score | Pbo (−0.35 vs. −0.25; p = 0.0168) |
| Daily abd | No treatment differences between 125 mg vs. |
| pain/discomfort | Pbo |
| | Stage 1: 125 mg vs. Pbo (−0.35 vs. −0.13; |
| | p = 0.0170) |
| Days/wk with urgency | No treatment differences between 125 vs. Pbo |
| Days/wk with fecal | Numerical differences for 125 mg vs. |
| incontinence | Pbo (−0.96 vs. −0.55; p = 0.0643) |
| BMs/day | No treatment differences between 125 mg vs. |
| | Pbo |

FIG. 1 and Table 13 show subjects with clinical response in the crossover to placebo-free phase of the safety population. PC=Placebo-controlled Phase and PF=Placebo-free Phase. This data shows that subjects previously on placebo had a sharp increase in efficacy when they were crossed-over onto 125 mg b.i.d. crofelemer. This data also demonstrates that crofelemer efficacy continued to rise with length of use.

TABLE 13
Subjects with Clinical Response Crossover to Placebo-Free Phase, Safety
Population

| | | | Crofelemer | Crofelemer | Crofelemer |
|---|---|---|---|---|---|
| | | Placebo | 125 mg | 250 mg | 500 mg |
| Month | Statistic¹ | (N = 126) | (N = 99) | (N = 15) | (N = 12) |
| Month 1 | Responder - n/Ni (%) | 11/126 | 36/99 | 3/15 | 2/12 (16.7%) |
| | | (8.7%) | (36.4%) | (20.0%) | |
| | Odds Ratio (95% CI) | | 0.17 | | |
| | | | (0.09, 0.32) | | |
| | P-value (vs. Placebo in | | <.0001 | | |
| | PC) | | | | |
| Month 2 | Responder - n/Ni (%) | | 42/95 | 8/15 | 2/12 (16.7%) |
| | | | (44.2%) | (53.3%) | |
| | Odds Ratio (95% CI) | | 0.12 | | |
| | | | (0.06, 0.24) | | |
| | P-value (vs. Placebo in | | <.0001 | | |
| | PC) | | | | |
| Month 3 | Responder - n/Ni (%) | | 48/89 | 5/14 | 2/11 (18.2%) |
| | | | (53.9%) | (35.7%) | |
| | Odds Ratio (95% CI) | | 0.07 | | |
| | | | (0.03, 0.15) | | |
| | P-value (vs. Placebo in | | <.0001 | | |
| | PC) | | | | |
| Month 4 | Responder - n/Ni (%) | | 43/77 | 5/13 | 2/9 (22.2%) |
| | | | (55.8%) | (38.5%) | |
| | Odds Ratio (95% CI) | | 0.07 | | |
| | | | (0.03, 0.15) | | |
| | P-value (vs. Placebo in | | <.0001 | | |
| | PC) | | | | |
| Month 5 | Responder - n/Ni (%) | | 37/67 | 6/13 | 1/8 (12.5%) |
| | | | (55.2%) | (46.2%) | |
| | Odds Ratio (95% CI) | | 0.07 | | |
| | | | (0.03, 0.16) | | |
| | P-value (vs. Placebo in | | <.0001 | | |
| | PC) | | | | |

¹Ratio of responders and p value obtained from parameter estimates with effect for treatment and region.

TABLE 14
Subjects with Stool Consistency Response*

| | | | | |
|---|---|---|---|---|
| | | Crofelemer | Crofelemer | |
| Intent-to-Treat | Placebo | 125 mg | 250 mg | Crofelemer 500 mg |
| Population | (N = 138) | (N = 136) | (N = 54) | (N = 46) |

Stage 1

| | | | | |
|---|---|---|---|---|
| Responder | 11/50 (22.0%) | 13/44 (31.8%) | 20/54 | 23/46 (50.0%) |
| | | | (37.0%) | |
| Treatment Difference | | 9.80% | 15.00% | 28.00% |

Stage 2

| | | |
|---|---|---|
| Responder | 38/88 (43.2%) | 39/92 (42.4%) |
| Treatment Difference | | −0.80% |

Combined

| | | |
|---|---|---|
| Responder | 49/138 | 53/136 |
| | (35.5%) | (39.0%) |
| Treatment Difference | | 3.50% |

*<4 stool consistency score at least 2 of the 4 efficacy assessment weeks.

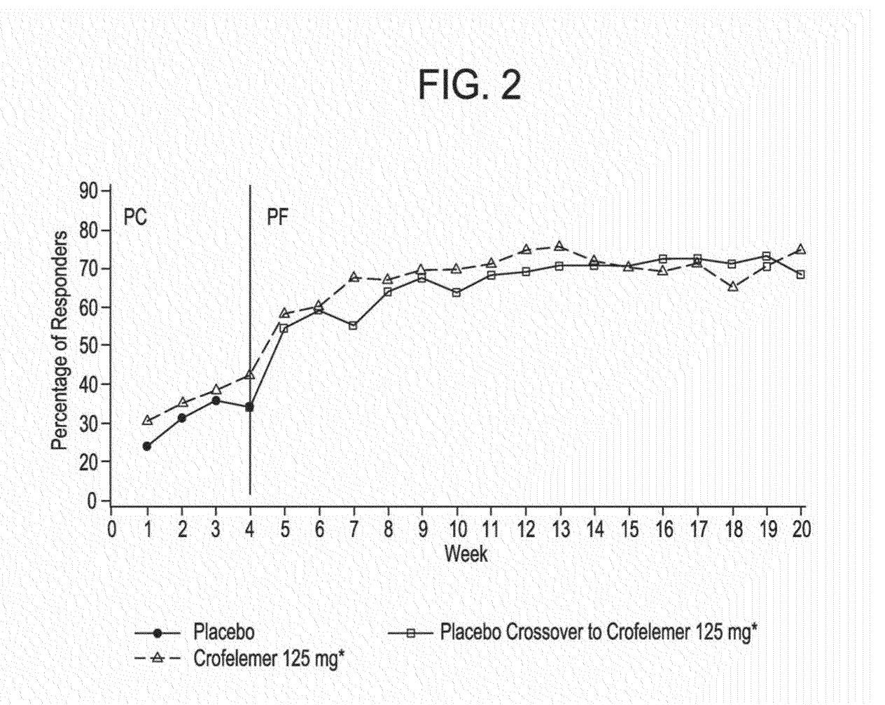

FIG. 2 and Table 14 show subjects with stool consistency response in the crossover to placebo-free phase in the safety population. PC=Placebo-controlled Phase and PF=Placebo-free Phase. This data shows that subjects previously on placebo had a sharp increase in efficacy when they were crossed-over onto 125 mg crofelemer. This data also demonstrates that crofelemer efficacy continued to rise with length of use.

TABLE 15
Subjects With Abnormal ECG Findings

| | | Crofelemer | Crofelemer | Crofelemer | All |
|---|---|---|---|---|---|
| Safety Population | Placebo | 125 mg | 250 mg | 500 mg | Crofelemer |
| Characteristic, n (%) | (N = 137) | (N = 130) | (N = 54) | (N = 42) | (N = 226) |
| Post Baseline Abnormal | 35 (25.5%) | 34 (26.2%) | 18 (33.3%) | 13 (31.0%) | 65 (28.8%) |
| ECG Finding¹ | | | | | |

QT Interval (msec)

| | | | | | |
|---|---|---|---|---|---|
| >450 with Baseline ≦450 | 2 (1.5%) | 4 (3.1%) | 1 (1.9%) | 0 | 5 (2.2%) |
| >480 with Baseline ≦480 | 1 (0.7%) | 0 | 1 (1.9%) | 0 | 1 (0.4%) |
| >500 with Baseline ≦500 | 0 | 0 | 1 (1.9%) | 0 | 1 (0.4%) |
| Change from Baseline 30-60 | 9 (6.6%) | 4 (3.1%) | 6 (11.1%) | 3 (7.1%) | 13 (5.8%) |
| Change from Baseline >60 | 4 (2.9%) | 1 (0.8%) | 2 (3.7%) | 0 | 3 (1.3%) |

QT Interval Linear Regression Correction
(msec)

| | | | | | |
|---|---|---|---|---|---|
| >450 with Baseline ≦450 | 2 (1.5%) | 4 (3.1%) | 1 (1.9%) | 0 | 5 (2.2%) |
| >480 with Baseline ≦480 | 1 (0.7%) | 0 | 1 (1.9%) | 0 | 1 (0.4%) |
| >500 with Baseline ≦500 | 0 | 0 | 1 (1.9%) | 0 | 1 (0.4%) |
| Change from Baseline 30-60 | 7 (5.1%) | 4 (3.1%) | 6 (11.1%) | 3 (7.1%) | 13 (5.8%) |
| Change from Baseline >60 | 4 (2.9%) | 1 (0.8%) | 2 (3.7%) | 0 | 3 (1.3%) |

QT Interval Fridericias's Correction (msec)

| | | | | | |
|---|---|---|---|---|---|
| >450 with Baseline ≦450 | 6 (4.4%) | 1 (0.8%) | 2 (3.7%) | 0 | 3 (1.3%) |
| >480 with Baseline ≦480 | 1 (0.7%) | 1 (0.8%) | 0 | 0 | 1 (0.4%) |
| >500 with Baseline ≦500 | 1 (0.7%) | 0 | 0 | 0 | 0 |
| Change from Baseline 30-60 | 3 (2.2%) | 5 (3.8%) | 2 (3.7%) | 1 (2.4%) | 8 (3.5%) |
| Change from Baseline >60 | 4 (2.9%) | 0 | 1 (1.9%) | 0 | 1 (0.4%) |

QT Interval Bazett's Correction (msec)

| | | | | | |
|---|---|---|---|---|---|
| >450 with Baseline ≦450 | 8 (5.8%) | 4 (3.1%) | 2 (3.7%) | 0 | 6 (2.7%) |
| >480 with Baseline ≦480 | 2 (1.5%) | 2 (1.5%) | 1 (1.9%) | 0 | 3 (1.3%) |
| >500 with Baseline ≦500 | 2 (1.5%) | 0 | 0 | 0 | 0 |
| Change from Baseline 30-60 | 7 (5.1%) | 8 (6.2%) | 3 (5.6%) | 1 (2.4%) | 12 (5.3%) |
| Change from Baseline >60 | 3 (2.2%) | 1 (0.8%) | 1 (1.9%) | 0 | 2 (0.9%) |

Table 15 demonstrates that crofelemer is safe to be given to subjects and that there are no QT Interval issues associated with use. This is surprising and advantageous over other molecules used to treat diarrhea and IBS, which are know to be associated with QT issues.

From the above data and Figures, it has been demonstrated that the proportion of clinical responders was significantly higher in the crofelemer 125 mg group compared with placebo (p=0.0096; Combined data). Subjects switching from placebo to crofelemer upon conclusion of the placebo-controlled phase achieved rates of clinical response of between 36.4% and 55.8% for each of the 5 months of the placebo-free phase (p<0.0001; for data collected prior to lock). Stool consistency scores improved significantly in subjects receiving 125 mg b.i.d. crofelemer compared with placebo (p=0.0168; Combined data). Days per week that subjects experienced fecal incontinence decreased in subjects receiving 125 mg b.i.d. crofelemer compared with placebo (p=0.0643; Combined data). It is also demonstrated that crofelemer was well tolerated and demonstrated a safety profile comparable to placebo and no clinically important differences in safety assessments have been identified.

TABLE 16
Dose-Ranging Study for treatment of d-IBS: All Randomized Subjects

| Design: | P2, R, DB, PC, Dose-Ranging Study for treatment of d-IBS | |
|---|---|---|
| Treatment: | 125, 250, 500 mg Crofelemer b.i.d. or Pbo for 12 weeks | |
| N: | 125 mg: 62, 250 mg: 60, 250 mg: 62, Pbo: 61 | |
| Endpoint | Daily stool consistency | |
| Outcome: | ROME Foundation Definition¹ | Month 1 = 2.6% |
| | Treatment Δ, 125 mg dose | Month 2 = 9.0% |
| | | Month 3 = 10.7% |
| | All 3 months: Odds Ratio | 1.51 (0.78, 2.92) |
| | FDA Definition² | Month 1 = 0.6% |
| | Treatment Δ, 125 mg dose | Month 2 = 2.2% |
| | | Month 3 = −4.4% |
| | All 3 months: Odds Ratio | 1.09 (0.54, 2.20) |

¹ROME Foundation Stool consistency weekly responder: Subjects with <25% days with loose or watery stools in a given week. Based on Ad Hoc Table 1.1
²FDA Stool consistency weekly responder: Subjects with a weekly average stool consistency score <4 (4 = Loose stool)

Table 16 demonstrates that crofelemer is an efficacious treatment for d-IBS. It also demonstrates that crofelemer is an efficacious treatment for treating abnormal stool consistency associated with d-IBS.

In some embodiments, crofelemer is administered to treat abnormal stool consistency associated with d-IBS for at least one month.

In some embodiments, crofelemer is administered to treat abnormal stool consistency associated with d-IBS from between one month and two months or longer. In some embodiments, crofelemer is administered to treat abnormal stool consistency associated with d-IBS from between about one month and about three months or longer.

In some embodiments, crofelemer is administered to treat abnormal stool consistency associated with d-IBS for at least one month at about 125 mg b.i.d. In some embodiments, crofelemer is administered to treat abnormal stool consistency associated with d-IBS from between one month and two months or longer at about 125 mg b.i.d. In some embodiments, crofelemer is administered to treat abnormal stool consistency associated with d-IBS from between one month and three months or longer at about 125 mg b.i.d.

TABLE 17
Dose-Ranging Study for treatment of d-IBS
All Randomized Female Subjects

| Design: | P2, R, DB, PC, Dose-Ranging Study for | |
|---|---|---|
| | treatment of d-IBS | |
| Treatment: | 125, 250, 500 mg Crofelemer b.i.d. or Pbo for | |
| | 12 weeks | |
| N (125 mg | 125 mg: 46, Pbo: 46 | |
| females): | | |
| Endpoint | Daily stool consistency | |
| Outcome: | ROME Foundation | Month 1 = 6.5% |
| | Definition¹ | |
| | Treatment Δ, 125 mg dose | Month 2 = 10.8% |
| | | Month 3 = 10.8% |
| | All 3 months: Odds Ratio | 1.88 (0.87, 4.06) |
| | FDA Definition² | Month 1 = −2.1% |
| | Treatment Δ, 125 mg dose | Month 2 = 2.2% |
| | | Month 3 = −4.4% |
| | All 3 months: Odds Ratio | 1.20 (0.52, 2.75) |

¹ROME Foundation Stool consistency weekly responder: Subjects with <25% days with loose or watery stools in a given week. Based on Ad Hoc Table 1.1
²FDA Stool consistency weekly responder: Subjects with a weekly average stool consistency score <4 (4 = Loose stool)

Table 17 demonstrates that crofelemer is an efficacious treatment for d-IBS, especially to treat d-IBS in females. It also demonstrates that crofelemer is an efficacious treatment for treating abnormal stool consistency associated with d-IBS, and especially to treat abnormal stool consistency associated with d-IBS in females.

In some embodiments, crofelemer is administered to treat abnormal stool consistency associated with d-IBS in females for at least one month at 125 mg b.i.d. In some embodiments, crofelemer is administered to treat abnormal stool consistency associated with d-IBS in females from between one month and two months at about 125 mg b.i.d. In some embodiments, crofelemer is administered to treat abnormal stool consistency associated with d-IBS in females from between one month and three months or longer at about 125 mg b.i.d.

TABLE 18
Dose-Ranging Study for treatment of d-IBS in females
All Randomized Subjects

| Design: | P2, R, DB, PC, Dose-Ranging Study for treatment | | |
|---|---|---|---|
| | of d-IBS in females | | |
| Treatment: | 125 mg Crofelemer b.i.d. or Pbo for 12 weeks | | |
| N: | 125 mg: 120, Pbo: 120 | | |
| Endpoint | Daily abdominal pain¹ | | |
| Outcome: | Placebo (N = 120) | 125 mg (N = 120) | P-value |
| Month 1 | 66 (55.0%) | 75 (62.5%) | 0.2316 |
| Month 2 | 61 (50.8%) | 82 (68.3%) | 0.0059 |
| Month 3 | 65 (54.2%) | 79 (65.8%) | 0.0662 |

| | |
|---|---|
| All 3 Months | Odds Ratio: 1.67 (1.03, 2.70); p = 0.0357 |

¹Abdominal pain weekly responder is defined as subjects with at least 30% improvement compared to baseline abdominal pain score in a given week

Table 18 demonstrates that crofelemer is an efficacious treatment for d-IBS, especially to treat d-IBS in females. It also demonstrates that crofelemer is an efficacious treatment for treating abdominal pain associated with d-IBS, and especially to treat abdominal pain associated with d-IBS in females.

In some embodiments, crofelemer is administered to treat d-IBS for at least one month.

In some embodiments, crofelemer is administered to treat d-IBS from between one month and two months or longer. In one embodiment, crofelemer is administered to treat d-IBS from between about one month and about three months or longer.

In some embodiments, crofelemer is administered to treat d-IBS for at least one month at about 125 mg b.i.d. In some embodiments, crofelemer is administered to treat d-IBS from between one month and two months or longer at about 125 mg b.i.d. In some embodiments, crofelemer is administered to treat d-IBS from between one month and three months or longer at about 125 mg b.i.d.

In some embodiments, crofelemer is administered to treat d-IBS in females for at least one month at 125 mg b.i.d. In some embodiments, crofelemer is administered to treat d-IBS in females from between one month and two months at about 125 mg b.i.d. In some embodiments, crofelemer is administered to treat d-IBS in females from between one month and three months or longer at about 125 mg b.i.d.

In some embodiments, crofelemer is administered to treat abdominal pain associated with d-IBS for at least one month.

In some embodiments, crofelemer is administered to treat abdominal pain associated with d-IBS from between one month and two months or longer. In some embodiments, crofelemer is administered to treat abdominal pain associated with d-IBS from between about one month and about three months or longer.

In some embodiments, crofelemer is administered to treat abdominal pain associated with d-IBS for at least one month at about 125 mg b.i.d. In some embodiments, crofelemer is administered to treat abdominal pain associated with d-IBS from between one month and two months or longer at about 125 mg b.i.d. In some embodiments, crofelemer is administered to treat abdominal pain associated with d-IBS from between one month and three months or longer at about 125 mg b.i.d.

In some embodiments, crofelemer is administered to treat abdominal pain associated with d-IBS in females for at least one month at 125 mg b.i.d. In some embodiments, crofelemer is administered to treat abdominal pain associated with d-IBS in females from between one month and two months at about 125 mg b.i.d. In some embodiments, crofelemer is administered to treat abdominal pain associated with d-IBS in females from between one month and three months or longer at about 125 mg b.i.d.

### Example 9

### Assessment of the Safety and Efficacy of Orally Administered Crofelemer in Aids Patients

This study was a randomized, double-blind, multicenter (2 study sites), placebo-controlled, parallel-group study designed to assess the efficacy and safety of crofelemer 500 mg beads in subjects with HIV-associated diarrhea.

The primary objectives of the study were to evaluate the safety and efficacy of orally administered crofelemer for 96 hours for the symptomatic treatment of diarrhea in AIDS patients. The secondary objectives were 1) to characterize stool chloride ion concentration and daily stool chloride output in AIDS patients with diarrhea, 2) to compare stool chloride ion concentration and daily stool chloride output in AIDS patients with diarrhea treated with crofelemer or placebo, and 3) to assess stool consistency in AIDS patients with diarrhea treated with crofelemer or placebo.

There were 3 assessment periods during the study: 1) a 24-hour in-patient screening period to ensure that the subjects met all of the study criteria, during which baseline stool weight was assessed; 2) a 4-day inpatient treatment period, during which all subjects received their assigned treatment 4 times per day (Days 1-4); subjects were discharged from the hospital after 96 hours of treatment if clinically stable); and 3) a follow-up visit 7-9 days after discharge from the hospital. The use of anti-diarrhea medication (ADM) was not allowed during the study.

Efficacy measurements included assessments of stool weight and frequency, abnormal stool frequency, DGIS, MORE, body weight, time to diarrhea recurrence, and number of early dropouts (prior to completion of 4 days of treatment).

Efficacy endpoints in this study were as follows:

An efficacy endpoint in this study was the change in total daily stool weight during the Treatment Period. The other efficacy endpoints of the study were abnormal stool frequency, defined as watery or soft stools (change in daily abnormal stool frequency), stool frequency (change in daily stool frequency), DGIS (change from baseline in DGIS for each day [Days 1 through 4]), stool chloride concentration (mg chloride/g stool weight; change in daily stool chloride concentration) and measure of relief scores, where MORE was the maximum of: a) time from the start of treatment period to the first abnormal stool, b) the maximum time between abnormal stools, or c) the time between the last abnormal stool and the end of the treatment period.

The DGIS was the daily sum of mean symptom scores for each of 7 symptoms (nausea, vomiting, abdominal pain and/or cramps, excess gas, urgency, tenesmus, and incontinence) scored 4 times per day. Symptoms were rated on a 4-point scale from 0=absent to 3=severe.

A total of 85 subjects were enrolled into the study at 2 study sites. Two of subjects in each of the 2 treatment groups withdrew before completion of the in-patient treatment period.

All randomized subjects (n=85) received at least 1 dose of study drug and were included in efficacy analyses.

TABLE 19
Demographics:

| | | | Crofelemer |
|---|---|---|---|
| Characteristic | Placebo | | 500 mg Beads |
| Category or Statistic | (n = 42) | | (n = 43) |
| Age, Years | | | |
| Mean (± SD) | 38.9 | (7.6) | 41.0 (8.8) |
| Median | 36 | | 40 |
| Min, Max | 27, 55 | | 21, 60 |
| Sex, n (%) | | | |
| Male | 41 | (97.6)) | 42 (97.7) |
| Female | 1 | (2.4) | 1 (2.3) |
| Race, n (%) | | | |
| Caucasian | 26 | (61.9) | 32 (74.4) |
| Hispanic | 7 | (16.7) | 6 (14.0) |
| African American | 7 | (16.7) | 5 (11.6) |
| Other | 2 | (5.0) | 0 |

Abbreviations:
ITT = intent-to-treat;
Max = maximum;
Min = minimum,
SD = standard deviation.

The number of unformed (i.e., soft or watery) stools/day at baseline (screening [Day 0]) and during the week prior to baseline, and disease severity are presented in Table 20. Mean (±SD) unformed stools/day at baseline was 5.5 (3.3) in the placebo group and 4.6 (2.6) in the crofelemer group. Most subjects had mild (3-4 stools/day) or moderate (5-8 stools/day) disease severity at baseline. Five subjects in the crofelemer group and 4 subjects in the placebo group had severe diarrhea (>9 stools/day).

TABLE 20
Baseline Diarrhea Assessments: (ITT Population)

| | | |
|---|---|---|
| | | Crofelemer |
| Characteristic | Placebo | 500 mg Beads |
| Category or Statistic | (n = 42) | (n = 43) |

Unformed Stools During 24 Hours Prior to First Dose of Study Drug
(Screening [Day 0]), Stools/Day

| | | |
|---|---|---|
| Mean (±SD) | 5.6 (2.9) | 4.6 (2.5) |
| Median | 5 | 4 |
| Min, Max | 1, 6 | 0, 10 |

Unformed Stools During the Week Prior to First Dose of Study Drug
(Screening [Day 0]), Stools/Day

| | | |
|---|---|---|
| Mean (±SD) | 5.5 (2.9) | 5.1 (2.3) |
| Median | 6 | 5 |
| Min, Max | 1, 15 | 2, 10 |

Diarrhea Severity, n (%)

| | | |
|---|---|---|
| Mild (3-4 stools/day) | 10 (40.0) | 15 (57.1) |
| Moderate (5-8 stools/day) | 13 (52.0) | 8 (30.8) |
| Severe (>9 stools/day) | 2 (8.0) | 3 (11.5) |

Abbreviations:
ITT = intent-to-treat;
Max = maximum;
Min = minimum,
SD = standard deviation.

Concomitant antiretroviral medications were received by majority of subjects (71 of 85): 36 of 42 (85.7%) in the placebo group and 35 of 43 (81.4%) in the crofelemer group. Protease inhibitors were taken by 69.4% of subjects. The concomitant use of antiretrovirals, including protease inhibitors, was balanced between groups.

The primary efficacy analysis was change in total daily stool weight during the 4-day in-patient treatment period. An endpoint of reduction in stool weight is an appropriate measure of the extent of watery diarrhea in patients with HIV-associated diarrhea due to high water content in the diarrhea experienced by these patients.

As shown in Table 21, there were significantly greater decreases in stool weight from baseline to Day 4 (last treatment day) in the crofelemer group compared with placebo (p=0.0335 by generalized linear model) in the ITT population. The repeated measures analysis of longitudinal data over the course of the 4-day Treatment Period did not show significant improvements in the crofelemer group compared to placebo; p=0.4108 for changes in total stool weight.

TABLE 21
Change in Stool Weight

| | | | |
|---|---|---|---|
| | | Crofelemer | |
| | Placebo | 500 mg Beads | p-value |
| In-Patient Period | (n = 42) | (n = 43) | (vs. Placebo)^{a} |

Stool Weight at Baseline (g)

| | | | |
|---|---|---|---|
| Mean (±SD) | 730.9 (720.14) | 861.3 (604.67) | 0.3832 |
| Median | 547.0 | 707.7 | |
| Min, Max | 206, 4701 | 220, 3407 | |

Categories of Stool Weight at Baseline, n (%)

| | | | |
|---|---|---|---|
| Low (≦740 g) | 28 (66.7) | 24 (55.8) | 0.2725 |
| High (>740 g) | 14 (33.3) | 19 (44.2) | |

Change in Stool Weight: Baseline to Day 4 (g)

| | | | |
|---|---|---|---|
| Mean (±SD) | −192.4 (381.57) | −401.3 (531.65) | 0.0335 |
| Median | −232.8 | −267.5 | |
| Min, Max | −1319, 683 | −1815, 854 | |

Abbreviations:
ITT = intent-to-treat;
Max = maximum;
Min = minimum,
SD = standard deviation.
^{a}P-value for baseline mean comparison is from generalized linear model with analysis center as a covariate. P-value for baseline percentage comparison is from CMH test with analysis center as a covariate. The estimates and p values are from the generalized linear model for the change from baseline result, with independent variables: treatment, analysis center, baseline category (value = Low for ≦740 g and High for >740 g in stool weight), and the interaction between treatment and baseline category (if p value >0.15, the interaction term was not included).

The effect of crofelemer in decreasing stool weight was more pronounced in the subgroup of subjects with baseline stool weight >740 g when compared to subjects with baseline stool weight ≦740 g (p-values for differences relative to placebo were 0.0202 [>740 g subgroup] versus 0.6820 [≦740 g subgroup]).

There were significantly greater decreases from baseline in stool weight at Day 3 in the crofelemer group compared with placebo (p=0.0128).

Significantly greater decreases in the frequency of abnormal stools (i.e., watery or soft stools) from baseline to Day 4 were observed in the crofelemer group compared with placebo (p=0.0069 by generalized-linear model) in the ITT population. The repeated measures analysis of longitudinal data over the course of the Treatment Period also indicates significantly greater reductions in the crofelemer group compared with placebo; p=0.0330 for changes in abnormal stool frequency. In addition, subjects in the crofelemer group had significantly greater decreases in abnormal stool frequency from baseline to Day 2 (p=0.0454) and from baseline to Day 3 (p=0.0064) compared with subjects in the placebo group.

TABLE 22
Abnormal Stool Frequency

| | | | |
|---|---|---|---|
| | | Crofelemer | |
| | Placebo | 500 mg Beads | p-value |
| In-Patient Period | (n = 42) | (n = 43) | (vs. Placebo)^{a} |

Abnormal Stool (soft or watery stool) Frequency at Baseline; abnormal
stools/day

| | | | |
|---|---|---|---|
| Mean (±SD) | 4.8 (2.12) | 4.9 (2.58) | 0.9933 |
| Median | 4.0 | 4.0 | |
| Min, Max | 3, 12 | 2, 14 | |

Categories of Abnormal Stool Frequency at Baseline, n (%)

| | | | |
|---|---|---|---|
| Low (≦5/day) | 30 (71.4) | 32 (74.4) | 0.7847 |
| High (>5/day) | 12 (28.6) | 11 (25.6) | |

Change in Abnormal Stool Frequency: Baseline to Day 4; abnormal
stools/day

| | | | |
|---|---|---|---|
| Mean (±SD) | −2.1 (1.94) | −2.8 (2.23) | 0.0069 |
| Median | −2.0 | −3.0 | |
| Min, Max | −6, 4 | −11, 2 | |

Abbreviations:
ITT = intent-to-treat;
Max = maximum;
Min = minimum,
SD = standard deviation.
^{a}P-value for baseline mean comparison is from generalized linear model with analysis center as a covariate. P-value for baseline percentage comparison is from CMH test with analysis center as a covariate. The estimates and p values are from the generalized linear model for the change from baseline result, with independent variables: treatment, analysis center, baseline category (value = Low for ≦5/day and High for >5/day in abnormal stool frequency), and the interaction between treatment and baseline category (if p value >0.15, the interaction term was not included).

The effect of crofelemer in decreasing abnormal stool frequency was more pronounced in the subgroup with high abnormal stool counts at baseline (>5/day) compared to the subgroup with low abnormal stool counts at baseline (≦5/day; p-values for differences relative to placebo were 0.0041 [>5/day subgroup] versus 0.8184 [≦5/day subgroup])

Significantly greater decreases in stool frequency (i.e., formed, watery, and soft stools) from baseline to Day 4 were observed in the crofelemer group compared with placebo (p=0.0046 by generalized-linear model) in the ITT population (Table 23). The repeated measures analysis of longitudinal data over the course of the Treatment Period also indicates significantly greater reductions in the crofelemer group compared with placebo; p=0.0236 for changes in stool frequency.

The effect of crofelemer in decreasing stool frequency was more pronounced in the subgroup with high stool counts at baseline (>5/day) compared to the subgroup with low stool counts at baseline (≦5/day) (p-values for differences relative to placebo were 0.0019 [>5/day subgroup] versus 0.7912 [≦5/day subgroup]).

TABLE 23
Stool Frequency

| | | | |
|---|---|---|---|
| | | Crofelemer | |
| | Placebo | 500 mg Beads | p-value |
| In-Patient Period | (n = 42) | (n = 43) | (vs. Placebo)^{a} |

Stool Frequency at Baseline; stools/day

| | | | |
|---|---|---|---|
| Mean (±SD) | 5.1 (2.22) | 5.0 (2.46) | 0.8442 |
| Median | 4.0 | 4.0 | |
| Min, Max | 3, 12 | 2, 14 | |

Categories of Stool Frequency at Baseline, n (%)

| | | | |
|---|---|---|---|
| Low (≦5/day) | 27 (64.3) | 32 (74.4) | 0.2850 |
| High (>5/day) | 15 (35.7) | 11 (25.6) | |

Change in Stool Frequency: Baseline to Day 4; stools/day

| | | | |
|---|---|---|---|
| Mean (±SD) | −1.7 (1.92) | −2.5 (2.45) | 0.0116 |
| Median | −2.0 | −2.5 | |
| Min, Max | −5, 5 | −10, 2 | |

Abbreviations:
ITT = intent-to-treat;
Max = maximum;
Min = minimum,
SD = standard deviation.
^{a}P-value for baseline mean comparison is from generalized linear model with analysis center as a covariate. P-value for baseline percentage comparison is from CMH test with analysis center as a covariate. The estimates and p values are from the generalized linear model for the change from baseline result, with independent variables: treatment, analysis center, baseline category (value = Low for ≦5/day and High for >5/day in stool frequency), and the interaction between treatment and baseline category (if p value >0.15, the interaction term was not included).

The crofelemer group had significantly greater decreases in stool frequency from baseline to Day 2 (p=0.0223) and from baseline to Day 3 (p=0.0140) compared with placebo.

### Daily Gastrointestinal Symptom Score

In a repeated measures analysis of longitudinal data over the course of the in-patient period (i.e., changes from baseline at each day during Days 1-4), a statistical trend indicating greater improvements in DGIS scores was observed in the crofelemer group compared with placebo (p=0.0559).

### Stool Chloride Concentrations

Stool chloride concentrations were measured in this study because the antisecretory, antidiarrheal effect of crofelemer is likely due to the inhibition of CFTR channel and CACC (Ca²⁺-activated chloride channel) in the GI lumen; this inhibition blocks luminal chloride ion (Cl⁻) secretion and accompanying high volume water loss in secretory diarrhea (Fischer et al. 2004. J Ethnopharmacol 93(2-3):351-357; Tradtrantip et al. 2010. Mol Pharmacol 77(1):69-78); thus reduced luminal Cl⁻ secretion should result in lower stool chloride concentrations. Subjects in the crofelemer group had significantly greater reductions in stool chloride concentrations from baseline to Day 4 when compared with placebo (p=0.0024 by generalized linear model) among subjects with stool chloride data (placebo n=25, crofelemer n=26). Mean (±SD) changes from baseline to Day 4 were 0.123 (0.7138) mg/g in the placebo group and −0.245 (0.5556) mg/g in the crofelemer group.

### Example 10

### Safety and Tolerability of Crofelemer 125 mg Bid for treatment of Noninfectious Diarrhea in HIV-Positive Subjects

The objective of this example was to assess the safety and tolerability of crofelemer 125 mg BID during double-blind and open-label conditions. Patient safety and tolerability data were analyzed from a Phase 3, randomized, double-blind, placebo-controlled, adaptive design trial that comprised a dose-selection stage (Stage I) and a dose-assessment stage (Stage II) as described above (Example 8). Each stage was comprised of a 4-wk placebo (PBO)-controlled phase and a 20-wk, PBO-free phase. An integrated summary of safety is also presented for all crofelemer-treated subjects (n=439) pooled from the Phase 3 study and an ongoing 48-week open-label safety study.

In the Phase 3 study safety population, demographic and baseline characteristics were similar between PBO (n=137) and crofelemer (CRO, n=130), as was person exposure years (PEY: 12.1 and 11.9, respectively). An endpoint, the proportion of subjects with ≦2 watery stools/week for ≧2 of 4 weeks, was significantly higher for CRO vs. PBO (p=0.0096). The percentage of subjects reporting any adverse event (AE) was comparable between placebo (32.8%) and CRO (34.6%). The most common AEs were gastrointestinal events and infections, and nearly all occurred in ≦2 subjects (see Table 24). In PBO-treated subjects (n=99, PEY=34.6) switched to active treatment, the rate of most common AEs was generally lower than that observed during the PBO-controlled phase (Table 24). Similar findings were observed for the integrated summary of safety (n=439, PEY=199.1) (Table 24). There was no deterioration of immune status in either study, as measured by mean CD4 counts and HIV viral load. Two deaths occurred (1 each in the PBO and CRO groups); neither was considered related to study drug.

TABLE 24
Most Common Adverse Events

| RCT PBO | RCT CRO | PBO Crossover^{a} | All CRO^{b} |
|---|---|---|---|
| (n = 137) | (n = 130) | (n = 99) | (n = 439) |
| PEY | | | |
| 12.1 | 11.9 | 34.6 | 199.1 |

Number of Subjects (Rate^{c})

GI Disorders

| | | | | |
|---|---|---|---|---|
| Dyspepsia | 0 (0) | 2 (17.0) | 0 (0) | 5 (2.5) |
| Flatulence | 0 (0) | 2 (16.9) | 3 (8.8) | 11 (5.6) |
| Abdominal pain | 1 (8.3) | 2 (16.9) | 2 (5.8) | 15 (6.2) |
| Haemorrhoids | 0 (0) | 2 (16.9) | 2 (5.8) | 7 (3.6) |

Infections

| | | | | |
|---|---|---|---|---|
| Upper respiratory | 4 (33.5) | 4 (42.8) | 1 (2.9) | 44 (23.5) |
| tract infection | | | | |
| Urinary tract | 1 (8.3) | 3 (25.3) | 0 (0) | 15 (7.8) |
| infection | | | | |

^{a}PBO subjects in RCT that crossed over to CRO 125 mg in 20-wk, PBO-free period
^{b}All CRO 125 mg subjects in RCT and subjects who received CRO 125 mg in 48-wk safety study
^{c}Event rate calculated as the number of events/100 PEY

In conclusion, crofelemer 125 mg BID significantly reduced non-infectious diarrhea in HIV+ subjects, and the safety profile was comparable to PBO in subjects treated for up to 48 weeks. These findings are consistent with crofelemer's localized activity in the gut and minimal systemic absorption.

### Example 11

### Clinical Pharmacology of Crofelemer for Treatment of Noninfectious Diarrhea in HIV Positive Subjects

The objective of these nonclinical and clinical studies was to characterize the clinical pharmacology and drug-drug interaction potential of crofelemer. The pharmacokinetics (PK) of crofelemer was evaluated in healthy subjects and HIV+ individuals using complete and sparse PK sampling methods, respectively. Membrane permeability and drug-drug interaction potential for crofelemer was evaluated in vitro using human liver microsomes (cytochrome P450 [CYP] inhibition), CYP induction (human hepatocytes), Caco-2 cells (permeability and P-glycoprotein), and transfected cells (MRP2, MRP4, OATP2B1, PEPT1, ASBT, OATB1A2). The population PK of six antiretroviral medications and measures of HIV status were assessed in HIV+ subjects receiving oral crofelemer 125 mg BID or placebo during a Phase 3 trial.

Crofelemer (CRO) systemic exposure was below the limit of quantification in plasma samples from the majority of both healthy and HIV+ subjects. In 46 healthy subjects, one had quantifiable crofelemer exposure (Cₘₐₓ=57 ng/mL, AUC=251 ng.h/mL). Of 229 subjects with HIV-associated diarrhea, only one sample from one subject had quantifiable crofelember exposure (71.7 ng/mL). These data are consistent with the negligible permeability observed in vitro, and the lack of clinically significant inhibition of CYP enzymes (IC₅₀=0.28-3.5 μM) or drug transporters (IC₅₀=7-50 μM). In vivo, population PK analysis showed no significant effects of crofelemer on the PK of ritanovir, tenofovir, emtricitabine, lamivudine, lopinavir, or efavirenz. Furthermore, no significant effects of crofelemer treatment on antiretroviral efficacy were observed as measured by the absence of clinically significant shifts in CD4, CD8, and HIV viral load.

Crofelemer 125 mg BID was minimally absorbed in healthy subjects and subjects with HIV, and there was no evidence of significant drug-drug interactions in vitro or in HIV+ subjects. In individuals with HIV-associated diarrhea taking multiple concomitant medications, crofelemer surprisingly may be administered without the risk of significant systemic exposure and a low risk of drug-drug interactions.

### Example 12

### Use of Crofelemer for Symptomatic Relief of Non-Infectious Diarrhea

Crofelemer in the form of delayed-release tablets is useful as an anti-diarrheal for the symptomatic relief of non-infectious diarrhea in adult patients with HIV/AIDS on anti-retroviral therapy. One 125 mg delayed-release tablet is taken orally twice a day, with or without food. The tablets should not be crushed or chewed. The tablets should be swallowed whole. In an exemplary embodiment, the tablet is a white, oval, enteric-coated 125 mg delayed-release tablet printed on one side with “125SLXP”. It is recommended that the tablets be stored at 20° C.-25° C. (68° F.-77° F.); excursions permitted between 15° C.-30° C. (59° F.-86° F.).

Inactive ingredients in the delayed-release tablet include, but are not limited to, microcrystalline cellulose, croscarmellose sodium, colloidal silicon dioxide, and magnesium stearate.

Coating ingredients of the delayed-release tablet include, but are not limited to, ethylacrylate and methylacrylate copolymer dispersion, talc, triethyl citrate, and white dispersion which contains xanthan gum, titanium dioxide, propyl paraben, and methyl paraben.

It is recommended that infectious etiologies of diarrhea are ruled out in patients before starting crofelemer. If infectious etiologies are not considered, there is a risk that patients with infectious etiologies will not receive the appropriate therapy and their disease may worsen.

The efficacy of crofelemer 125 mg delayed-release tablets twice daily was evaluated in a randomized, double-blind, placebo-controlled (one month) and placebo-free (five month), multi-center study. The study enrolled 374 HIV-positive patients on stable anti-retroviral therapy (ART) with a history of diarrhea for one month or more. Diarrhea was defined as either persistently loose stools despite regular use of anti-diarrheal medication (ADM) (e.g., loperamide, diphenoxylate, and bismuth subsalicylate) or one or more watery bowel movements per day without regular ADM use.

Patients were excluded if they had a positive gastrointestinal (GI) biopsy, GI culture, or stool test for multiple bacteria (Salmonella, Shigella, Campylobacter, Yersinia, Mycobacterium), bacterial toxin (Clostridium difficile), ova and parasites (Giardia, Entamoeba, Isospora, Cyclospora, Cryptosporidium, Microsporidium), or viruses (Cytomegalovirus). Patients were also excluded if they had a history of ulcerative colitis, Crohn's disease, celiac sprue (gluten-enteropathy), chronic pancreatitis, malabsorption, or any other GI disease associated with diarrhea.

The study had a two-stage adaptive design. In both stages, patients received placebo for 10 days (screening period) followed by randomization to crofelemer or placebo for 31 days of treatment (double-blind period). Only patients with 1 or more watery bowel movements per day on at least 5 of the last 7 days in the screening period were randomized to the double-blind period. Each stage enrolled patients separately. The dose for the second stage was selected based on an interim analysis of data from the first stage. In the first stage, patients were randomized 1:1:1:1 to one of three crofelemer dose regimens (125, 250, or 500 mg twice daily) or placebo. In the second stage, patients were randomized 1:1 to crofelemer 125 mg twice daily or placebo. The efficacy analysis was based on results from the double-blind portion of both stages.

Each study stage also had a five month period (placebo-free period) that followed the double-blind period. Patients treated with crofelemer continued the same dose in the placebo-free period. In the first stage, patients that received placebo were re-randomized 1:1:1 to one of the three crofelemer dose regimens (125, 250, or 500 mg twice daily) in the placebo-free period. In the second stage, patients that received placebo were treated with crofelemer 125 mg twice daily in the placebo-free period.

The median time since diagnosis of HIV was 12 years. The percentage of patients with a CD4 cell count of less than 404 was 39%. The percentage of patients with a HIV viral load greater than or equal to 1000, 400 to 999, and less than 400 HIV copies/mL was 7%, 3%, and 9%, respectively; the remainder had a viral load that was not detectable. The median time since diarrhea started was 4 years. The median number of daily watery bowel movements was 2.5 per day.

In the double-blind period of the study, 136 patients received crofelemer 125 mg twice daily, 54 patients received 250 mg twice daily, 47 patients received 500 mg twice daily, and 138 patients received placebo. The percentages of patients that completed the double-blind period were 92%, 100%, 85%, and 94% in the 125 mg, 250 mg, 500 mg, and placebo arms, respectively.

Most patients received concomitant protease inhibitors (PI) during the double-blind period (Table 25). The most frequently used anti-retroviral therapies (ARTs) in each group were tenofovir/emtricitabine, ritonavir, and lopinavir/ritonavir.

TABLE 25
Concomitant ART Use in the Double-Blind Period

| | 125 mg | 250 mg | 500 mg | Placebo |
|---|---|---|---|---|
| | BID | BID | BID | BID |
| | (N = 136) | (N = 54) | (N = 46) | (N = 138) |
| | n (%) | n (%) | n (%) | n (%) |
| Any ART | 135 (99) | 53 (98) | 45 (98) | 134 (97) |
| Any PI | 87 (64) | 41 (76) | 33 (72) | 97 (70) |
| Tenofovir/Emtricitabine | 45 (33) | 22 (41) | 16 (35) | 52 (38) |
| Ritonavir | 46 (34) | 18 (33) | 15 (33) | 49 (36) |
| Lopinavir/Ritonavir | 30 (22) | 21 (39) | 15 (33) | 40 (29) |
| Efavirenz/Tenofovir/ | 30 (22) | 7 (13) | 7 (15) | 21 (15) |
| Emtricitabine | | | | |
| Tenofovir disoproxil | 18 (13) | 8 (15) | 5 (11) | 14 (10) |
| fumarate | | | | |
| Antazanavir sulfate | 19 (14) | 3 (6) | 6 (13) | 22 (16) |
| Abacavir w/ lamivudine | 17 (13) | 5 (9) | 5 (11) | 18 (13) |
| Darunavir | 19 (14) | 4 (7) | 4 (9) | 14 (10) |
| Raltegravir | 16 (12) | 4 (7) | 5 (11) | 11 (8) |
| Valaciclovir | 12 (9) | 8 (15) | 5 (11) | 16 (12) |
| hydrochloride | | | | |
| Fosamprenavir | 12 (9) | 6 (11) | 4 (9) | 13 (9) |
| Zidovudine w/ | 12 (9) | 3 (6) | 3 (7) | 15 (11) |
| lamivudine | | | | |
| Lamivudine | 7 (5) | 6 (11) | 4 (9) | 6 (4) |
| Nevirapine | 8 (6) | 6 (11) | 3 (7) | 9 (7) |
| Atazanavir | 5 (4) | 6 (11) | 2 (4) | 2 (1) |

Abbreviations:
ART = antiretroviral therapy;
PI = Protease Inhibitor;
BID = twice daily.
* greater than 10% of Any Treatment Group

An efficacy endpoint was the proportion of patients with a clinical response, defined as less than or equal to 2 watery bowel movements per week during at least 2 of the 4 weeks of the placebo-controlled phase. Patients who received concomitant anti-diarrhea medications (ADMs) or opiates were counted as clinical non-responders.

A significantly larger proportion of patients in the crofelemer 125 mg twice daily group experienced clinical response compared with patients in the placebo group (17.6% vs. 8.0%, 1 sided p<0.01).

In the randomized clinical study, examination of duration of diarrhea, baseline number of daily watery bowel movements, use of protease inhibitors, CD4 cell count and age subgroups did not identify differences in the consistency of the crofelemer treatment effect among these subgroups. There were too few female subjects and subjects with an HIV viral load >400 copies to adequately assess differences in effects in these populations. Among race subgroups, there were no differences in the consistency of the crofelemer treatment effect except for the subgroup of African-Americans; crofelemer was less effective in African-Americans than non-African-Americans.

Although the CD4 cell count and HIV viral load did not appear to change over the one month placebo-controlled period, the clinical significance of this finding is unknown because of the short duration of the placebo-controlled period.

A small number of clinical responders to crofelemer (125 mg twice daily) entered the placebo-free period (n=22); 16 were responding at the end of month 3, and 14 were responding at the end of month 5.

Most common adverse reactions (incidence ≧3%) are upper respiratory tract infection, bronchitis, cough, flatulence and increased bilirubin. In three placebo-controlled trials, a total of 696 HIV-positive patients received crofelemer in the form of delayed-release tablets for a mean duration of 78 days. Of the total population across the three trials, 229 patients received a dose of 125 mg twice a day for a mean duration of 141 days, 69 patients received a dose of 250 mg twice a day for a mean duration of 139 days, 102 patients received a dose of 250 mg four times a day for a mean duration of 14 days, 54 patients received a dose of 500 mg twice a day for a mean duration of 146 days, and 242 patients received a dose of 500 mg four times a day for a mean duration of 14 days. Adverse reactions to the crofelemer tablets 125 mg twice a day that occurred in at least 2% of patients and at a higher incidence than placebo are provided in Table 26.

TABLE 26
Adverse Reactions Occurring in at Least 2% of Patients in the 125 mg
Twice Daily Group

| | Crofelemer 125 mg | |
|---|---|---|
| | BID* | Placebo |
| | N = 229 | N = 274 |
| Adverse Reaction | n (%) | n (%) |
| Upper respiratory tract infection | 13 (5.7) | 4 (1.5) |
| Bronchitis | 9 (3.9) | 0 |
| Cough | 8 (3.5) | 3 (1.1) |
| Flatulence | 7 (3.1) | 3 (1.1) |
| Increased bilirubin | 7 (3.1) | 3 (1.1) |
| Nausea | 6 (2.6) | 4 (1.5) |
| Back pain | 6 (2.6) | 4 (1.5) |
| Arthralgia | 6 (2.6) | 0 |
| Urinary tract infection | 5 (2.2) | 2 (0.7) |
| Nasopharyngitis | 5 (2.2) | 2 (0.7) |
| Musculoskeletal pain | 5 (2.2) | 1 (0.4) |
| Hemorrhoids | 5 (2.2) | 0 |
| Giardiasis | 5 (2.2) | 0 |
| Anxiety | 5 (2.2) | 1 (0.4) |
| Increased alanine aminotransferase | 5 (2.2) | 3 (1.1) |
| Abdominal distension | 5 (2.2) | 1 (0.4) |

*Twice daily

Adverse events reported in greater than or equal to 1% and less than 2% of patients taking crofelemer delayed-release tablets, and that occurred at a higher incidence than placebo, were dizziness, muscle spasms, myalgia, and pain in extremity. Adverse reactions were similar in patients who received doses greater than 250 mg daily.

In vitro studies have shown that crofelemer has the potential to inhibit cytochrome P450 isoenzyme 3A and transporters MRP2 and OATP1A2 at concentrations expected in the gut. Due to the minimal absorption of crofelemer, it is unlikely to inhibit cytochrome P450 isoenzymes 1A2, 2A6, 2B6, 2C9, 2C19, 2D6, 2E1 and CYP3A4 systemically.

In a specific embodiment, administration of crofelemer delayed-release tablets did not have a clinically relevant interaction with nelfinavir, zidovudine, or lamivudine in a drug-drug interaction trial.

Based on animal data, the delayed-release crofelemer tablets may cause fetal harm. Reproduction studies performed with crofelemer in rats at oral doses up to 177 times the recommended daily human dose of 4.2 mg/kg revealed no evidence of impaired fertility or harm to the fetus. In pregnant rabbits, crofelemer at an oral dose of about 96 times the recommended daily human dose of 4.2 mg/kg, caused abortions and resorptions of fetuses. However, it is not clear whether these effects are related to the maternal toxicity observed. A pre- and postnatal development study performed with crofelemer in rats at oral doses of up to 177 times the recommended daily human dose of 4.2 mg/kg revealed no evidence of adverse pre- and postnatal effects in offspring. There are, however, no adequate, well-controlled studies in pregnant women. Because animal reproduction studies are not always predictive of human response, this drug should be used during pregnancy only if clearly needed.

No dose modifications are recommended with respect to CD4 cell count and HIV viral load, based on the findings in subgroups of patients defined by CD4 cell count and HIV viral load. The safety profile of crofelemer was similar in patients with baseline CD4 cell count less than 404 cells/μL (lower limit of normal range) (N=388) and patients with baseline CD4 cell counts greater than or equal to 404 cells/μL (N=289). In addition, the safety profile of crofelemer was similar in patients with baseline HIV viral loads less than 400 copies/mL (N=412) and patients with baseline HIV viral loads greater than or equal to 400 copies/mL (N=278).

Consistent with the mechanism of action of crofelemer (i.e., inhibition of CFTR and CaCC in the gastrointestinal lumen), data suggest stool chloride concentrations decreased in patients treated with crofelemer delayed-release tablets for four days relative to placebo. Stool chloride concentrations were decreased in both African American patients and non-African American patients.

At a dose 10 times the maximum recommended dose, crofelemer does not appear to prolong the QTc interval to any clinically relevant extent.

The absorption of crofelemer is minimal following oral dosing in healthy adults and HIV positive patients and concentrations of crofelemer in plasma are below the level of quantitation (50 ng/mL). Therefore, standard pharmacokinetic parameters such as area under the curve, maximum concentration, and half-life have not been estimated.

No metabolites of crofelemer have been identified in healthy subjects or patients in clinical trials.

Administration of a single 500 mg dose of crofelemer with a high-fat meal was not associated with an increase in systemic exposure of crofelemer in healthy volunteers. In a previous randomized, double-blind, parallel group, placebo-controlled, two-stage trial (ADVENT), crofelemer was administered at least one-half hour before the morning and evening meals. Therefore, crofelemer can be administered with or without a meal.

Results of a crossover study in healthy volunteers showed crofelemer 500 mg administered four times daily for five days had no effect on the exposure of zidovudine and nelfinavir when administered as a single dose. A 20% decrease in lamivudine exposure was also observed in the same study but was not considered to be clinically important.

Crofelemer was negative in the bacterial reverse mutation assay, chromosomal aberration assay, and rat bone marrow micronucleus assay.

Crofelemer, at oral doses of up to 738 mg/kg/day (177 times the recommended human daily dose of 4.2 mg/kg), had no effects on fertility or reproductive performance of male and female rats.

### Example 13

### Characterization of Interactions Between Crofelemer and Antiretroviral Drug

Herein we (1) characterize the interaction between crofelemer and antiretroviral drugs and (2) characterize the effect of antiretroviral drugs on viral load if interaction between crofelemer and antiretroviral drugs exists. Data were analyzed from a Phase 3, randomized, double-blind, placebo-controlled, adaptive design trial that comprised a dose-selection stage (Stage I) and a dose-assessment stage (Stage II) as described above (Example 8). Each stage was comprised of a 4-wk placebo (PBO)-controlled phase and a 20-wk, PBO-free phase.

Blood samples were obtained for the characterization of crofelemer pharmacokinetics and the examination of steady-state concentrations of antiretroviral drugs that subjects were taking. Subjects were sampled at five visits. HIV-1 viral load, CD4 count, and CD8 count were obtained at Visit 0 to establish eligibility for the trial and repeated at Visit 1 to minimize the effects of intra-subject baseline variability. HIV-1 viral load samplings were subsequently repeated after study drug treatment at Visits 3 and 8. Blood samples were also obtained at Visit 0, 1, 3, 8 for pre-crofelemer dose antiretroviral and crofelemer concentration levels. At Visit 1, pre-dose samples were obtained before the administration of crofelemer.

The analyses were based on the data obtained from both stages. Three hundred and fifty-three subjects were included in the analysis dataset who had baseline concentration record plus at least one post dose antiretroviral (ARV) concentration record. The majority of these subjects (97.5%) had at least three antiretroviral drug combination treatments before the start of the study. There were 126 different combinations of antiretroviral treatments within the 353 subjects. Due to the limitation of small overall count of most of antiretroviral measures, the analyses were conducted on the antiretroviral drugs that had concentration data points in at least 25% of subjects. Six antiretroviral drugs satisfied this criterion, and they included ritonavir (RTV), tenofovir (TNF), emtricitabine (FTC), lamivudine (3TC), lopinavir (LPV), and efavirenz (EFV). Most of the patients in the trial were treated with these ARV drugs. Therefore, the analyses were conducted for the six medications mentioned above. Table 27 lists the abbreviation corresponding to each antiretroviral drug. Table 28 lists the top six antiretroviral drug combinations, with the most used ARV combination being EFV, FTC and TNF. Table 29 shows the list of antiretroviral maintained treatment combination which had at least five subjects, and Table 30 indicates all ARV combinations used in the study. There were 25 ARV combinations that had more than three subjects, and more than 80% ARV combinations that had only two subjects or less.

TABLE 27
List of Abbreviations for Each Antiviral Drug

| Antiretroviral Drug | Abbreviation |
|---|---|
| ABACAVIR | ABC |
| ATAZANAVIR | ATV |
| DARUNAVIR | DRV |
| DIDANOSINE | DDI |
| EFAVIRENZ/ENFUVIRTIDE | EFV |
| EMTRICITABINE | FTC |
| ETRAVIRINE | ETR |
| FOSAMPRENAVIR | APV |
| INDINAVIR | IDV |
| LAMIVUDINE | 3TC |
| LOPINAVIR | LPV |
| MARAVIROC | MVC |
| NELFINAVIR | NFV |
| NEVIRAPINE | NVP |
| RALTEGRAVIR | RAL |
| RITONAVIR | RTV |
| SAQUINAVIR | SQV |
| STAVUDINE | d4T |
| TENOFOVIR | TNF |
| TIPRANAVIR | TPV |
| ZIDOVUDINE | ZDV |

TABLE 28
Top Six Antiretroviral Combinations by Subject Count

| ARV Combination | N |
|---|---|
| EFV, FTC, TNF | 60 |
| FTC, LPV, RTV, TNF | 33 |
| ATV, FTC, RTV, TNF | 25 |
| APV, FTC, RTV, TNF | 15 |
| DRV, FTC, RTV, TNF | 12 |
| 3TC, ABC, LPV, RTV | 10 |

TABLE 29
Subject Count by Antiretroviral Combination (n ≧ 5)

| Antiretroviral combination | Count |
|---|---|
| EFV, FTC, TNF | 60 |
| FTC, LPV, RTV, TNF | 33 |
| ATV, FTC, RTV, TNF | 25 |
| APV, FTC, RTV, TNF | 15 |
| DRV, FTC, RTV, TNF | 12 |
| 3TC, ABC, LPV, RTV | 10 |
| 3TC, EFV, ZDV | 6 |
| 3TC, ABC, ATV, RTV | 6 |
| FTC, NVP, TNF | 5 |
| DDI, LPV, RTV, TNF | 5 |
| 3TC, NFV, ZDV | 5 |
| 3TC, LPV, RTV, ZDV | 5 |
| 3TC, ABC, ZDV | 5 |

TABLE 30
All Antiretroviral Combinations

| Antiretroviral combination | Count |
|---|---|
| EFV, FTC, TNF | 60 |
| FTC, LPV, RTV, TNF | 33 |
| ATV, FTC, RTV, TNF | 25 |
| APV, FTC, RTV, TNF | 15 |
| DRV, FTC, RTV, TNF | 12 |
| 3TC, ABC, LPV, RTV | 10 |
| 3TC, EFV, ZDV | 6 |
| 3TC, ABC, ATV, RTV | 6 |
| FTC, NVP, TNF | 5 |
| DDI, LPV, RTV, TNF | 5 |
| 3TC, NFV, ZDV | 5 |
| 3TC, LPV, RTV, ZDV | 5 |
| 3TC, ABC, ZDV | 5 |
| FTC, RAL, TNF | 4 |
| 3TC, NVP, ZDV | 4 |
| 3TC, LPV, RTV, TNF | 4 |
| 3TC, ABC, LPV, RTV, TNF | 4 |
| 3TC, ABC, ATV | 4 |
| LPV, RTV | 3 |
| DRV, EFV, FTC, RTV, TNF | 3 |
| ABC, LPV, RTV, TNF | 3 |
| 3TC, ATV, RTV, ZDV | 3 |
| 3TC, APV, RTV, ZDV | 3 |
| 3TC, ABC, EFV | 3 |
| 3TC, ABC, APV, RTV | 3 |
| NVP, RAL | 2 |
| LPV, RTV, SQV | 2 |
| FTC, LPV, RTV, TNF, ZDV | 2 |
| FTC, LPV, RTV, SQV, TNF | 2 |
| ETR, FTC, RAL, TNF | 2 |
| DRV, ETR, RAL, RTV | 2 |
| ATV, FTC, RAL, RTV, TNF | 2 |
| ATV, FTC, LPV, RTV, TNF | 2 |
| APV, FTC, TNF | 2 |
| ABC, FTC, LPV, RTV, TNF | 2 |
| 3TC, NVP, TNF | 2 |
| 3TC, MVC, RAL, TNF | 2 |
| 3TC, DRV, RAL, RTV | 2 |
| 3TC, ATV, ZDV | 2 |
| 3TC, ABC, NVP, TNF | 2 |
| 3TC, ABC, NFV | 2 |
| 3TC, ABC, LPV, RTV, ZDV | 2 |
| 3TC, ABC, DRV, RTV | 2 |
| 3TC, ABC, APV | 2 |
| LPV, RTV, TNF, ZDV | 1 |
| LPV, RTV, TNF | 1 |
| FTC, TNF | 1 |
| FTC, RAL, RTV, TNF, TPV | 1 |
| FTC, NFV, TNF | 1 |
| FTC, LPV, RTV, d4T | 1 |
| FTC, LPV, RAL, RTV, TNF, ZDV | 1 |
| FTC, LPV, RAL, RTV, TNF | 1 |
| FTC, LPV, NVP, RTV, TNF | 1 |
| ETR, LPV, RAL, RTV | 1 |
| ETR, FTC, TNF | 1 |
| EFV, FTC, RTV, TNF, TPV, ZDV | 1 |
| EFV, FTC, RAL, TNF | 1 |
| EFV, FTC, LPV, RTV, TNF | 1 |
| EFV, FTC, LPV, RAL, RTV, TNF | 1 |
| EFV, EFV, FTC, FTC, TNF, TNF | 1 |
| DRV, RTV, TNF | 1 |
| DRV, RTV | 1 |
| DRV, RAL, RTV | 1 |
| DRV, NVP, RAL, RTV | 1 |
| DRV, MVC, RAL, RTV | 1 |
| DRV, FTC, RTV, TNF, d4T | 1 |
| DRV, FTC, RTV, SQV, TNF | 1 |
| DRV, FTC, RAL, RTV, TNF | 1 |
| DRV, FTC, RAL, RTV | 1 |
| DRV, ETR, RTV | 1 |
| DRV, ETR, FTC, RTV, TNF | 1 |
| DRV, ETR, FTC, RAL, RTV, TNF, ZDV | 1 |
| DRV, EFV, RTV | 1 |
| DDI, RTV, SQV, TNF | 1 |
| DDI, LPV, RTV, ZDV | 1 |
| DDI, DRV, FTC, RTV, TNF | 1 |
| ATV, RTV, ZDV | 1 |
| ATV, RTV, TNF | 1 |
| ATV, RAL, RTV | 1 |
| ATV, NVP, RTV, TNF | 1 |
| ATV, NVP, RTV, SQV | 1 |
| ATV, NVP, RTV | 1 |
| ATV, LPV, RTV | 1 |
| ATV, FTC, RTV, SQV, TNF | 1 |
| ATV, FTC, NVP, RTV, TNF | 1 |
| ATV, DRV, ETR, FTC, RAL, RTV, TNF | 1 |
| APV, RTV | 1 |
| APV, LPV, RTV, TNF | 1 |
| APV, FTC, LPV, RTV, TNF | 1 |
| APV, EFV, FTC, RTV, TNF | 1 |
| APV, DDI, EFV, FTC, RTV, TNF | 1 |
| ABC, NVP, d4T | 1 |
| ABC, LPV, RTV, d4T | 1 |
| ABC, EFV, LPV, RTV, TNF | 1 |
| ABC, DRV, EFV, RTV, TNF | 1 |
| ABC, DDI, NFV | 1 |
| ABC, ATV, RTV, TNF | 1 |
| ABC, ATV, RTV | 1 |
| ABC, APV, NVP | 1 |
| ABC, APV, FTC | 1 |
| 3TC, RTV, SQV, ZDV | 1 |
| 3TC, NVP, RTV, SQV, d4T | 1 |
| 3TC, NFV, d4T | 1 |
| 3TC, LPV, RAL, RTV, ZDV | 1 |
| 3TC, LPV, NVP, RTV | 1 |
| 3TC, LPV, MVC, RAL, RTV, TNF | 1 |
| 3TC, FTC, LPV, RTV, TNF, ZDV | 1 |
| 3TC, EFV, TNF | 1 |
| 3TC, DRV, RTV, ZDV | 1 |
| 3TC, DRV, MVC, RAL, RTV, TNF | 1 |
| 3TC, ATV, d4T | 1 |
| 3TC, ATV | 1 |
| 3TC, APV, ZDV | 1 |
| 3TC, APV, RTV | 1 |
| 3TC, APV, EFV, FTC, RTV, TNF, ZDV | 1 |
| 3TC, ABC, RAL | 1 |
| 3TC, ABC, LPV, RTV, TNF, ZDV | 1 |
| 3TC, ABC, LPV, RAL, RTV, TNF | 1 |
| 3TC, ABC, LPV, NVP, RTV, TNF | 1 |
| 3TC, ABC, IDV | 1 |
| 3TC, ABC, ETR, RAL | 1 |
| 3TC, ABC, EFV, NVP | 1 |
| 3TC, ABC, ATV, RTV, TNF, ZDV | 1 |
| 3TC, ABC, ATV, LPV, RTV, TNF, ZDV | 1 |
| 3TC, ABC, APV, RTV, TNF | 1 |
| 3TC, ABC, APV, DDI, RTV | 1 |

Given the intersubject variability in the pharmacokinetics of ARV drugs, concentrations of ARV drugs in the absence of crofelemer (Visit 0 or Visit 1) were used to define the baseline ARV drug concentration ratio to which other ARV drug concentration ratios on Visit 3 and Visit 8 were compared. The use of drug concentration ratios for the comparisons, therefore, took into account intersubject variability. For stage I, change from baseline was computed using the single-blind placebo-screening period (Visit 0) as the reference baseline value. If Visit 0 value was not available, then Visit 1 value (the run-in period) was used as the baseline value. For stage II, only Visit 1 value was available to be used as the baseline value. Every baseline record that was below the limit of detection (BLD) was replaced with 1.5 ng/mL for ratio calculation.

Concentrations of ARV drugs were assumed to be at steady state because subjects were maintained on these drugs before enrolling in the study. The ARV concentration data used in the analysis are observational data collected during the Phase 3 trial (Example 8). It was also assumed that antiretroviral drugs have substantial intersubject pharmacokinetic variability (Moyle & Gazzard, 1996, Drugs 51: 701-702; Hsu et al, 1998, Clinical Pharmacokinet 35:275-291).

Data visualization was used to examine the relationship between covariates and subject-level ARV concentration values. Due to the sparseness of the concentration data of antiretroviral drugs, the use of a multi-step exploratory approach was employed in analyzing the data.

Taking intrasubject variability into account, the baseline “placebo” (crofelemer-free ARV drug concentration) ratio was calculated by dividing crofelemer-free ARV drug concentration on Visit 1 with that on Visit 0. Since stage II data had visit 1 as the baseline visit, the placebo group analysis in terms of ratios was done using Stage I data only.

The Stage I (dose-selection) data consisted of data collected during the 10+4 days of single-blind, placebo screening phase; followed by data collected after randomization from the 31-day, double-bind, placebo-controlled treatment phase. In other words, the crofelemer-free antiretroviral concentration values were obtained from visits below:

Stage I:
placebo arm: Visits 0, 1, and 3, and
active crofelemer arms: Visits 0 and 1 (before the initiation of crofelemer dosing).

Stage II:
Visits 1 (before the initiation of crofelemer dosing)

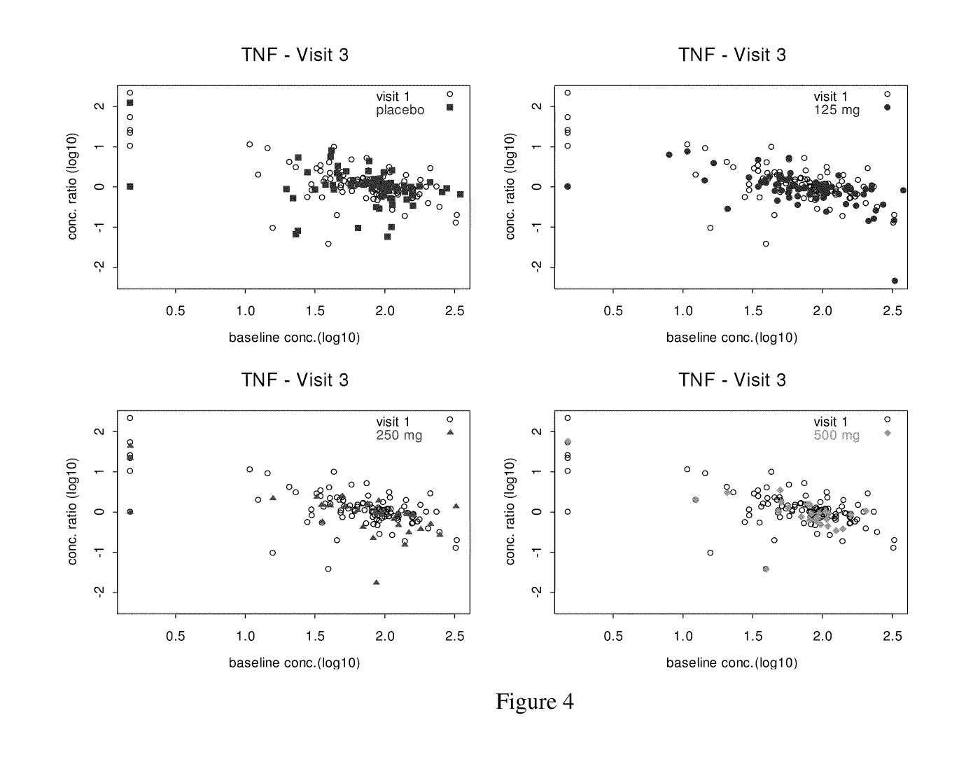

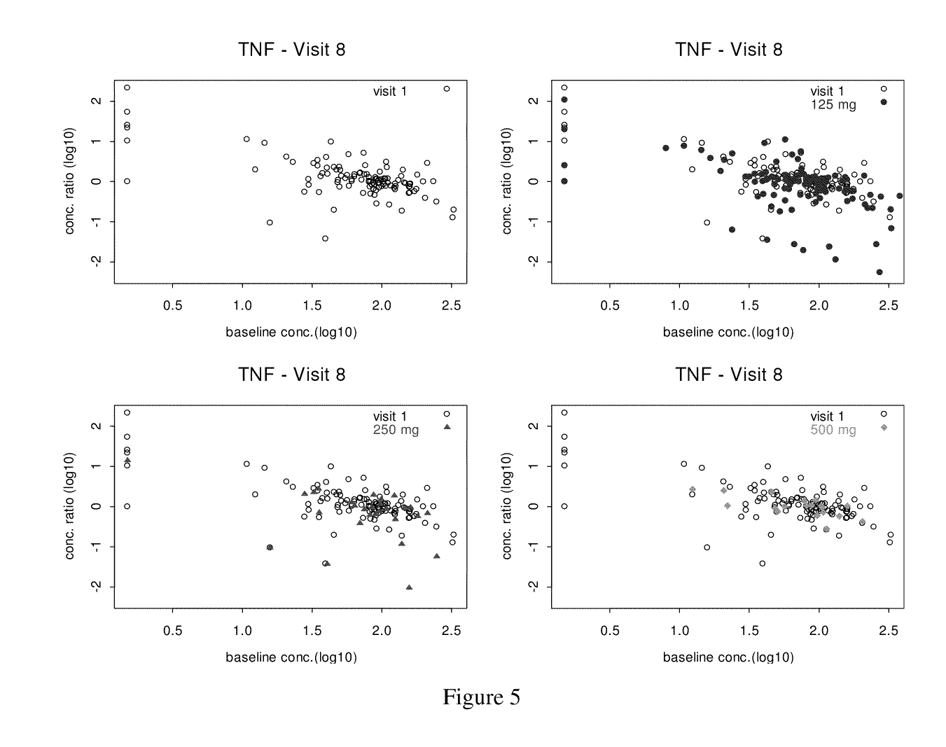

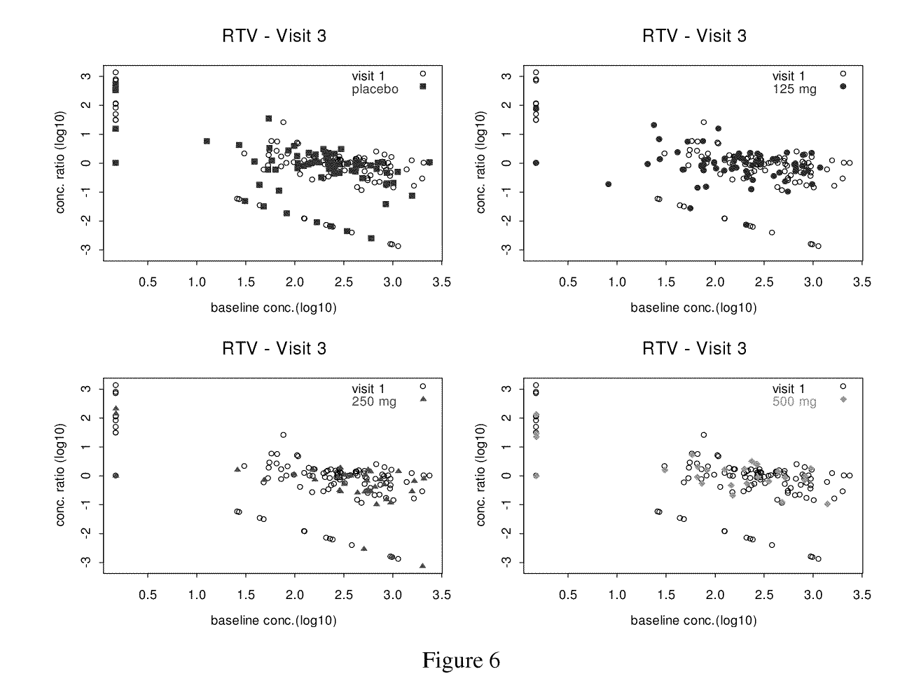

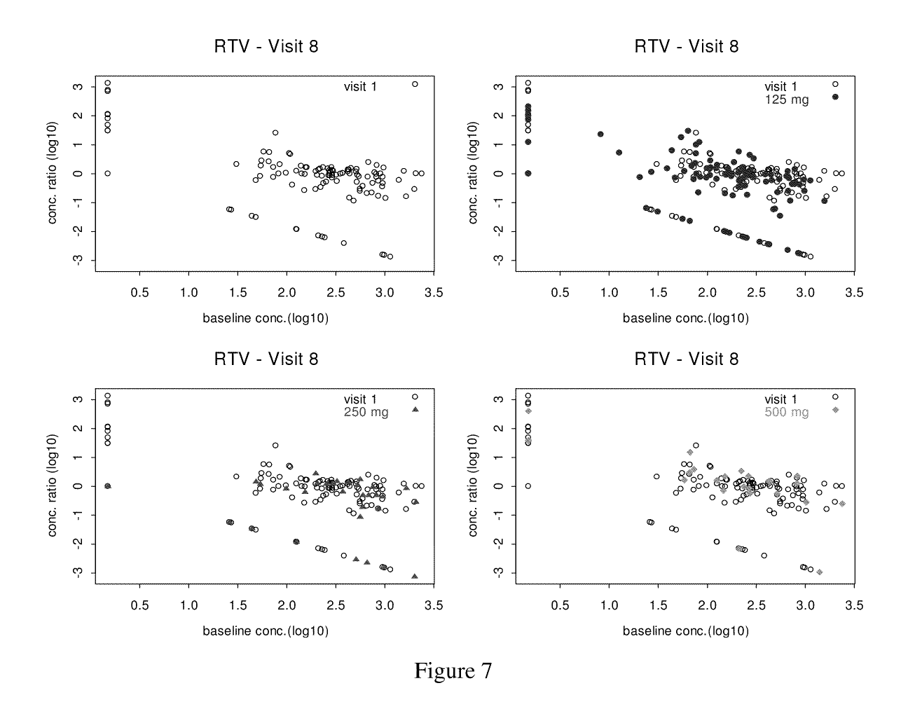

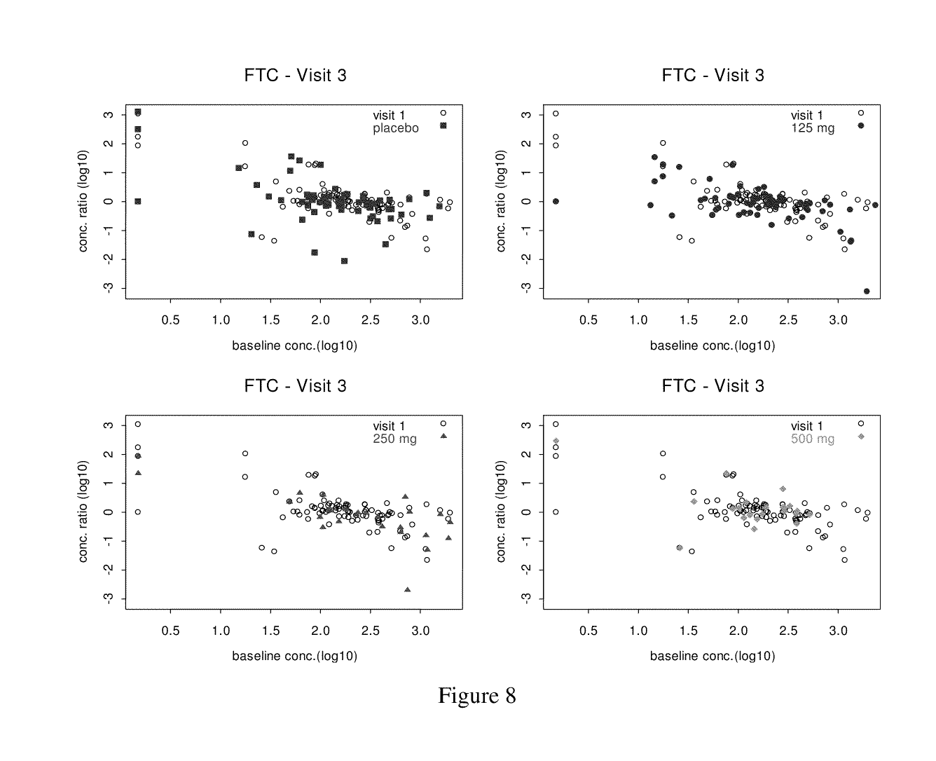

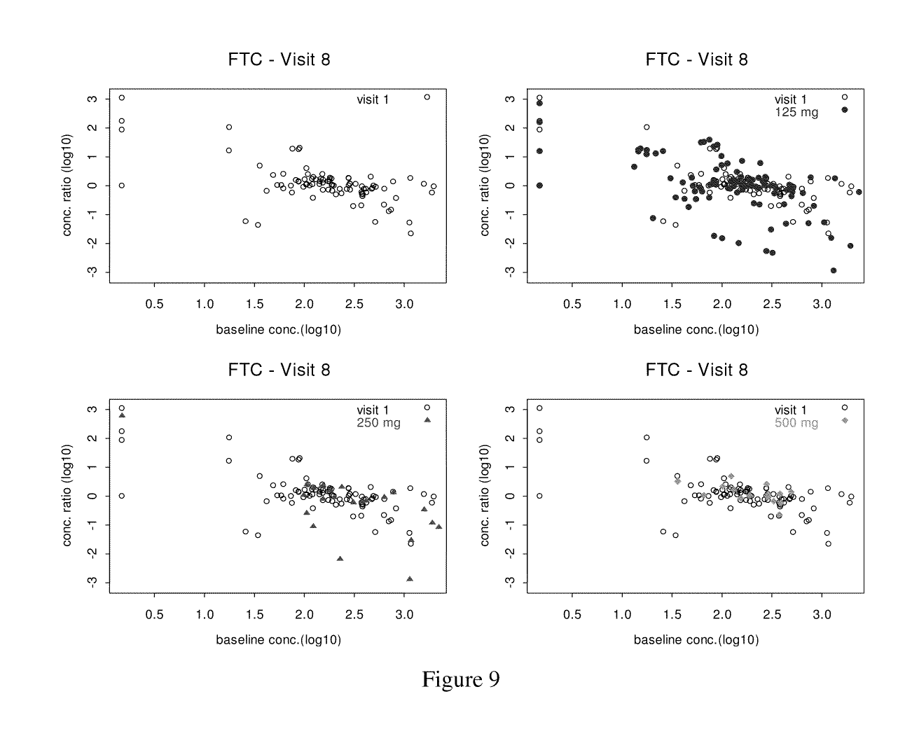

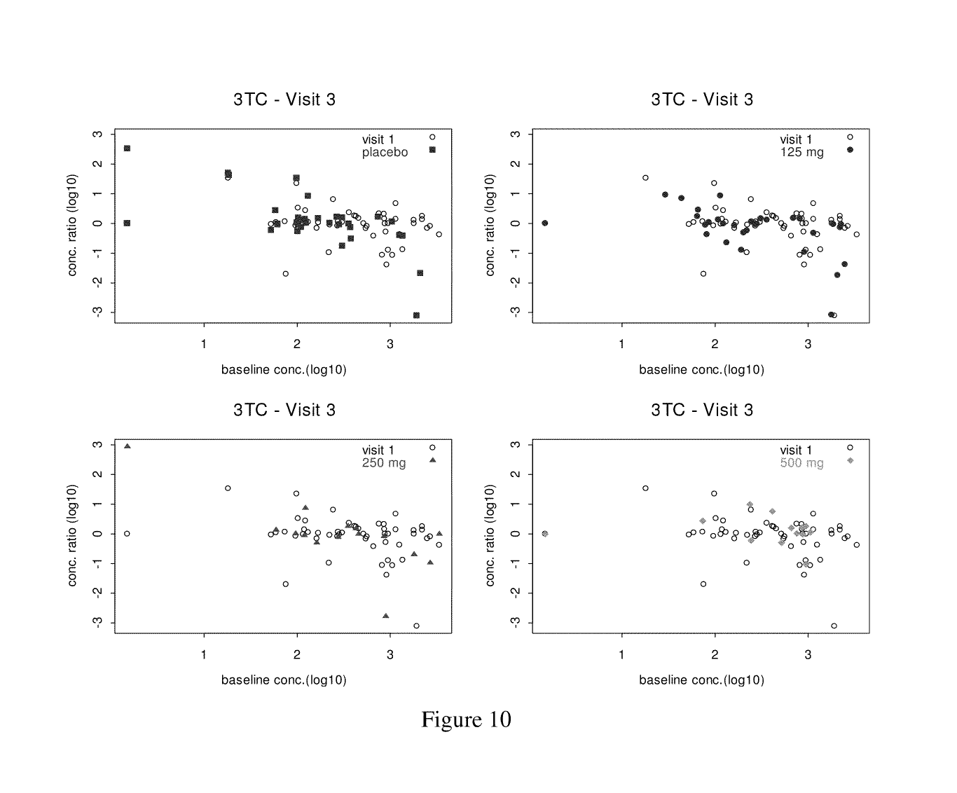

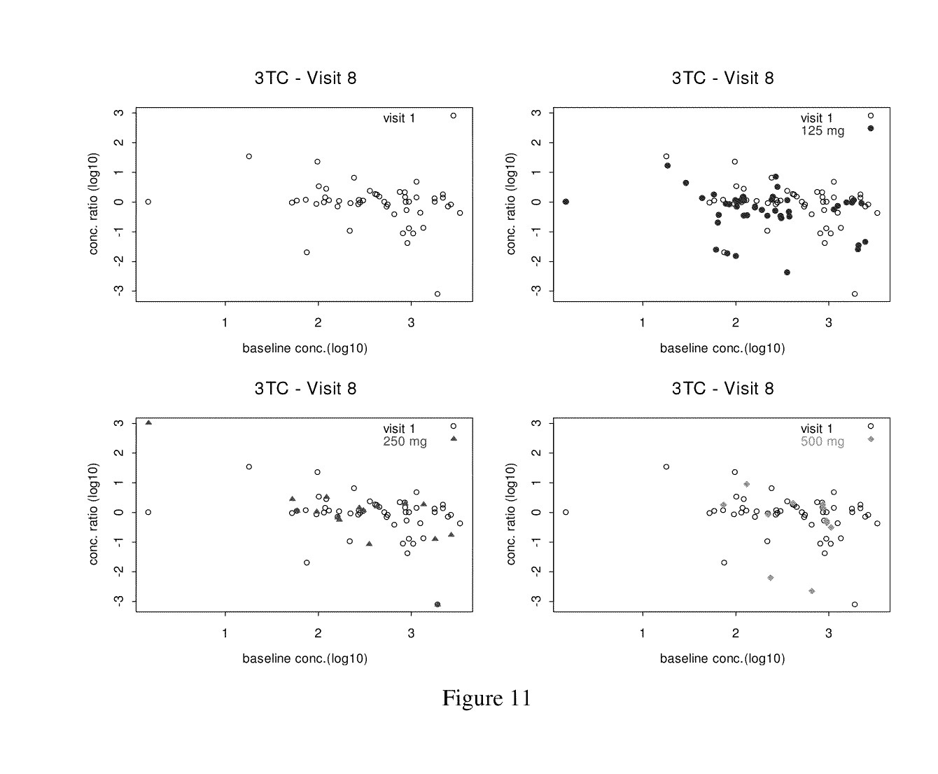

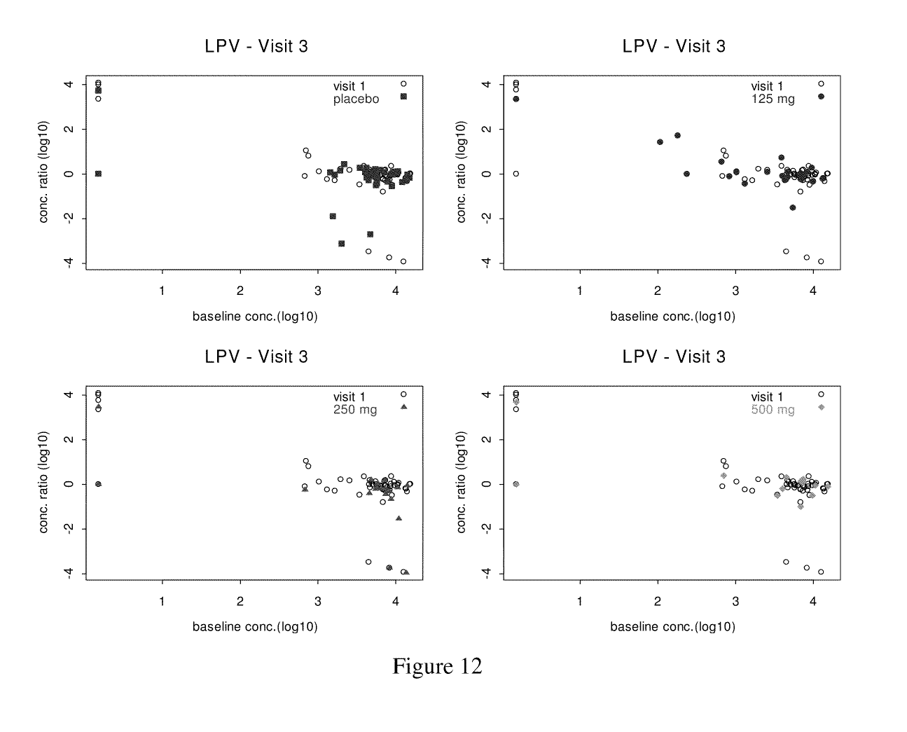

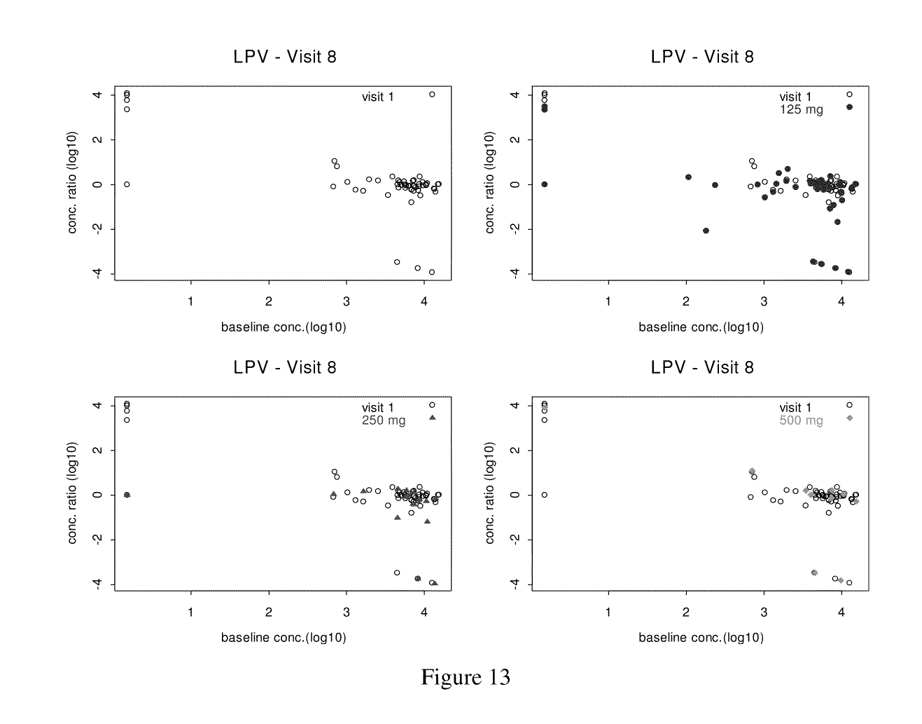

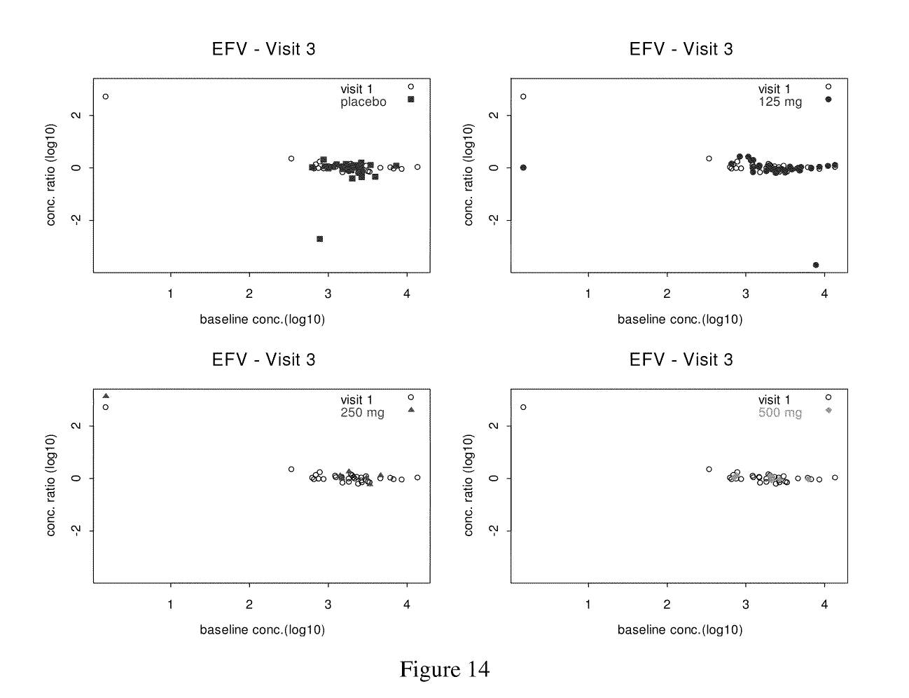

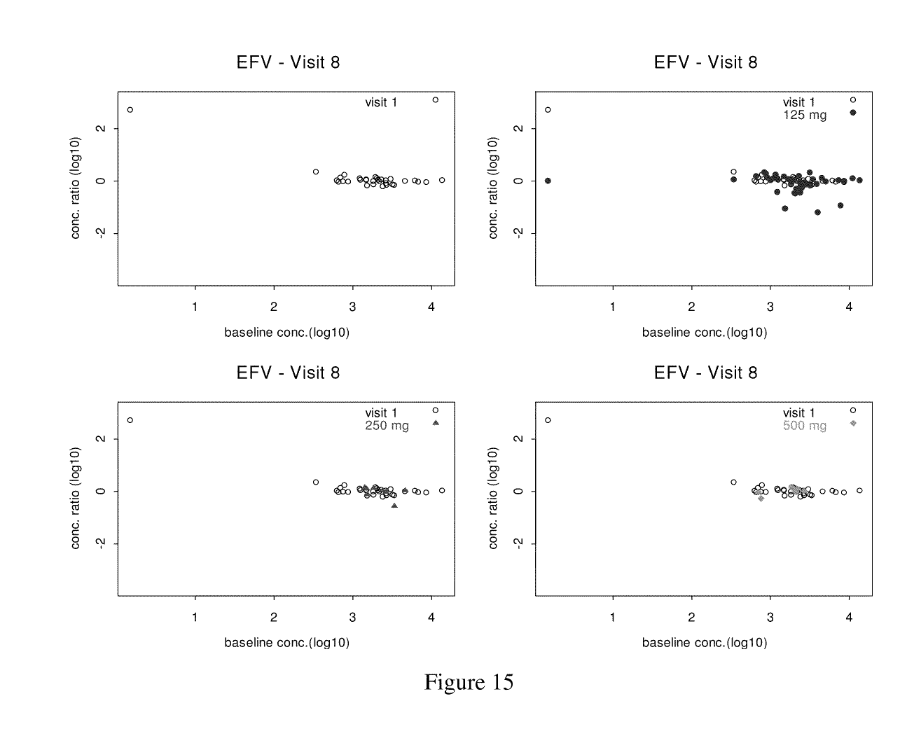

To characterize any possible interaction between crofelemer and antiretroviral drug, the changes from the baseline was calculated as ratios for each post-crofelemer (CFL) visit (which refers to visits and sample collections after initiation of crofelemer therapy). FIGS. 4, 6, 8, 10, 12 and 14 are graphical representations of visit 3 concentration ratios for various antiretroviral drugs (TNF, RTV, FTC, 3TC, LPV, EFV, respectively) overlaid on crofelemer-free concentration ratios (baseline concentration ratios) for the same drugs. The x-axis is log-transformed baseline concentration values, and y-axis is log-transformed antiretroviral concentration ratios (in the presence of placebo or crofelemer treatment). All four arms of Visit 3 clearly overlapped mostly with the Baseline Visit. FIGS. 5, 7, 9, 11, 13 and 15 illustrate the Visit 8 concentration ratios for TNF, RTV, FTC, 3TC, LPV and EFV, respectively, that were overlaid on crofelemer-free concentration ratios (baseline concentration ratios) for the same drugs in terms of log-transformed scale in both axes. The left upper panels of FIGS. 5, 7, 9, 11, 13 and 15 have CFL-free data only. The right upper panel of each of these panels had more data points in the 125 mg b.i.d. active arm since it is the selected dose administered in the Stage II. The panels show good overlap between CFL-free and each of the active CFL arms.

Changes of antiretroviral drug concentration were expressed as the ratio of concentration of the ith antiretroviral drug for the jth subject and kth visit divided by the baseline concentration of the ith antiretroviral drug for the jth subject. The baseline concentration value used was the single-blind placebo-screening period (Visit 0) from which to compute any change in value. If Visit 0 value was not available, then Visit 1 value (the run-in period) was used as the baseline value. Based on an initial analysis, low baseline subjects had a different ratio pattern than the rest of subjects. Considering the overall cluster pattern in the initial analysis, the general division criteria that can be applied using antiretroviral concentration baseline value are, log transformed baseline ≦0.5 (low baseline value) and the other for log transformed baseline concentration >0.5 (rest of the subjects).

Since some distributions of calculated ratio did not show a normal distribution even after log-transformation, a nonparametric Wilcoxon Rank test was used to compare the group means between placebo (CFL-free) and active group (CFL) by visit. To guarantee strict control of the familywise error rate when tests were not necessarily independent, multiplicity was accounted for using the Bonferroni correction approach.

There were four comparisons for each antiretroviral drug:
(1) Visit 3 ratios obtained for low baseline subjects vs. baseline ratios obtained for low baseline subjects;
(2) Visit 3 ratios obtained for higher baseline subjects vs. baseline ratios obtained for higher baseline subjects;
(3) Visit 8 ratios obtained for low baseline subjects vs. baseline ratios obtained for low baseline subjects; and
(4) Visit 8 ratios obtained for higher baseline subjects vs. baseline ratios obtained for higher baseline subjects.

The null hypothesis was there was no pharmacokinetic difference between placebo (CFL-free) and active group (CFL). A one sided test was used for the low baseline subjects, since the log-transformed ratio was bounded by zero. The alternative hypothesis was that log-transformed ratios for Visit 3 or Visit 8 were greater than the ones for the baseline visit. The hypothesis for the higher baseline subjects was two-sided.

Table 31 tabulates Bonferroni adjusted p-values. Based on Bonferroni-adjusted p-values exceeding 0.05 being classified as having no statistical significance, there is no statistically significant effect of crofelemer on the six ARV drug steady-state concentrations.

TABLE 31
Subject Count by Antiretroviral Combination (n ≧ 5)

| Bonferroni | | | | |
|---|---|---|---|---|
| adjusted p-value | Visit 3 | | Visit 8 | |
| Antiretroviral | Log(baseline) | Log(baseline) | Log(baseline) | Log(baseline) |
| Name | >0.5 | ≦0.5 | >0.5 | ≦0.5 |
| TNF | 0.094 | 1.000 | 0.088 | 1.000 |
| RTV | 1.000 | 1.000 | 1.000 | 1.000 |
| FTC | 0.746 | 0.758 | 1.000 | 1.000 |
| 3TC | 1.000 | 1.000 | 0.330 | 1.000 |
| LPV | 1.000 | 1.000 | 1.000 | 1.000 |
| EFV | 1.000 | NA | 1.000 | NA |

NA: no test for subject count <3

In confirmation with what was previously observed, quantifiable levels of crofelemer were not observed in the study (Example 8) from which reported ARV drug levels were used in this analysis.

In addition, from the results of the exploratory modeling (FIGS. 4-15) and statistical analysis (Table 31), crofelemer had no significant effect on the pharmacokinetics of the ARV drugs investigated. Because crofelemer had no significant effect on the pharmacokinetic of ARV drugs, its effect on viral load in the HIV patients was not investigated. This is because the pharmacokinetic of ARV drugs drive their pharmacodynamic effects.

In conclusion, crofelemer concentrations were not quantifiable. Crofelemer had no statistically significant effect on the steady-state concentrations of six antiretroviral drugs on which most of the patients in the study were maintained. Therefore, crofelemer therapy does not affect the pharmacokinetics, hence the pharmacodynamics of the antiretroviral drugs examined in this analysis.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating HIV-associated or highly active antiretroviral therapy (HAART)-associated diarrhea in an HIV positive subject, comprising
administering a composition comprising crofelemer to the subject,
wherein the composition has minimal drug-drug interactions with at least one other compound concurrently administered to the subject to treat an HIV infection.

2. A method of treating HIV-associated or highly active antiretroviral therapy (HAART)-associated diarrhea in an HIV positive subject, comprising:
administering a composition comprising crofelemer to the subject,
wherein the composition does not significantly inhibit the activity of at least one other compound concurrently administered to the subject to treat an HIV infection.

3. The method of claim 1, wherein the at least one other compound is an anti-retroviral therapy (ART) compound.

4. The method of claim 1, wherein the at least one other compound is selected from the group of: ritonavir, tenofovir, emtricitabine, lamivudine, lopinavir and efavirenz.

5. The method of claim 1, wherein the at least one other compound concurrently administered to the subject comprises efiravenz, emtricitabine and tenofovir.

6. The method of claim 1, wherein the at least one other compound concurrently administered to the subject comprises emtricitabine, ritonavir and tenofovir.

7. The method of claim 6, wherein the at least one other compound further comprises at least one selected from the group of: lopinavir, atazanavir, fosamprenavir, and darunavir.

8. The method of claim 1, wherein the at least one other compound concurrently administered to the subject comprises emtricitabine, lopinavir, ritonavir and tenofovir.

9. The method of claim 1, wherein the at least one other compound concurrently administered to the subject comprises lamivudine, lopinavir and ritonavir.

10. The method of claim 9, wherein the at least one other compound further comprises at least one selected from the group of: abacavir and zidovudine.

11. The method of claim 1, wherein the at least one other compound concurrently administered to the subject comprises lopinavir and ritonavir.

12. The method of claim 11, wherein the at least one other compound further comprises at least one selected from the group of: emtricitabine, tenofovir, lamivudine, abacavir, zidovudine and didanosine.

13. The method of claim 1, wherein the at least one other compound concurrently administered to the subject comprises: lamivudine and zidovudine.

14. The method of claim 13, wherein the at least one other compound further comprises at least one selected from the group of: efiravenz, nelfinavir, ritonavir, lopinavir and abacavir.

15. A method of treating HIV-associated or highly active antiretroviral therapy (HAART)-associated diarrhea in an HIV positive subject, comprising:
administering a composition comprising crofelemer to the subject,
wherein the composition does not significantly inhibit a CYP enzyme in vivo.

16. A method of treating HIV-associated or highly active antiretroviral therapy (HAART)-associated diarrhea in an HIV positive subject, comprising:
administering a composition comprising crofelemer to the subject,
wherein the composition does not significantly inhibit the activity of a drug transporter in vivo.

17. A method of treating HIV-associated or highly active antiretroviral therapy (HAART)-associated diarrhea in an HIV positive subject, comprising:
administering a composition comprising crofelemer to the subject,
wherein the composition does not significantly affect the efficacy of at least one other compound concurrently administered to the subject to treat an HIV infection.

18. A method of treating HIV-associated or highly active antiretroviral therapy (HAART)-associated diarrhea in an HIV positive subject, comprising:
administering a composition comprising crofelemer to the subject,
wherein the composition has negligible permeability as measured by in vitro permeability assays.

19. A method of treating HIV-associated or highly active antiretroviral therapy (HAART)-associated diarrhea in an HIV positive subject, comprising:
administering a composition comprising crofelemer to the subject,
wherein the composition has limited systemic exposure in vivo.

20. A method of treating HIV-associated or highly active antiretroviral therapy (HAART)-associated diarrhea in an HIV positive subject, comprising:
administering a composition comprising crofelemer to the subject,
wherein the composition does not provoke a significant adverse event in vivo.

21. The method of claim 20, wherein the adverse event is at least one selected from the group of: dyspepsia, flatulence, abdominal pain, hemorrhoids, upper respiratory tract infection, and urinary tract infection.

22. A method of treating HIV-associated or highly active antiretroviral therapy (HAART)-associated diarrhea in an HIV positive subject, comprising:
administering a composition comprising crofelemer to the subject,
wherein the composition does not cause deterioration of immune status in the subject.

23. The method of claim 1, wherein crofelemer is administered to the subject at a dosage of about 250 mg per day.

24. The method of claim 1, wherein crofelemer is administered to the subject at a dosage of about 125 mg two times per day.

25. The method of claim 1, wherein the composition is administered to the subject for between about one month and about six months.

26. The method of claim 1, wherein the composition is administered to the subject for between about three days and about six months.

27. The method of claim 1, wherein the subject experiences an improvement of symptoms on Day 3 after treatment begins.

28. The method of claim 1, wherein the composition is administered to the subject for at least about eight days.

29. The method of claim 1, wherein the composition is administered to the subject for between about eight days and about 24 weeks.

30. The method of claim 1, wherein the composition is administered to the subject for about six months.

31. The method of claim 1, wherein the composition is administered to the subject for at least about six months.

32. The method of claim 1, wherein the composition is administered to the subject for the duration of the subject's HIV infection.

33. The method of claim 1, wherein administration of the composition results in improvement in stool consistency.

34. The method of claim 1, wherein administration of the composition results in an improvement in a score for daily stool consistency relative to baseline measurements.

35. The method of claim 1, wherein administration of the composition results in alleviation of watery diarrhea.

36. The method of claim 1, wherein administration of the composition results in a decrease in the number of bowel movements per day relative to baseline measurements.

37. The method of claim 1, wherein administration of the composition results in a decrease in the number of water bowel movements per day relative to baseline measurements.

38. The method of claim 1, wherein administration of the composition results in an improvement in the daily abdominal score for pain or discomfort relative to baseline measurements.

39. The method of claim 1, wherein administration of the composition results in a decrease in the number of days per week that the subject experiences urgency relative to baseline measurements.

40. The method of claim 1, wherein administration of the composition results in a decrease in the number of days per week that the subject experiences fecal incontinence relative to baseline measurements.

41. The method of claim 1, wherein the subject has previously been administered protease inhibitors.
